(19) United States
(12) Patent Application Publication
(10) Pub. No.: US 2006/0150264 A1
(43) Pub. Date: Jul. 6, 2006

(54) Differential gene expression in schizophrenia

(76) Inventors: Sabine Bahn, Cambridge (GB); Margaret Ryan, Cambridge (GB); Stephen Huffaker, Cambridge (GB)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/560,279

(22) PCT Filed: Jun. 14, 2004
(86) PCT No.: PCT/GB04/02503

§ 371 Date: Mar. 10, 2006

(30) Foreign Application Priority Data
Jun. 13, 2003 (GB) 0313721.3
Jun. 13, 2003 (GB) 0313724.7

(51) Int. Cl. A01K 67/027; C12Q 1/68; G01N 33/567

(52) U.S. Cl. 800/18; 435/6; 435/7.2

(57) ABSTRACT

The invention relates to methods of identifying potential therapeutic agents for the prevention, treatment, or amelioration of schizophrenia, to methods of diagnosis of schizophrenia, to methods of identifying patients most likely to respond to a particular therapeutic treatment, to methods for selecting participants in clinical trials, and to methods of prevention, treatment, or amelioration of schizophrenia.

This invention relates to methods of identifying potential therapeutic agents for the prevention, treatment, or amelioration of schizophrenia (SZ), to methods of diagnosis of schizophrenia, to methods of identifying patients most likely to respond to a particular therapeutic treatment, to methods for selecting participants in clinical trials, and to methods of prevention, treatment, or amelioration of schizophrenia.

SZ is a severe psychiatric disorder characterized by hallucinations, delusions, disorganized thought, and various cognitive impairments. Polygenic models of inheritance and linkage analysis studies have postulated that several genes confer susceptibility to SZ. Hakak et al (PNAS, 2001, 98 (8) 4746-4751) have reported that the expression levels of genes involved in neuronal myelination, development, synaptic plasticity, neurotransmission, and signal transduction were altered in the dorsolateral prefrontal cortex of SZ brain tissue. Mimmack et al (PNAS, 2002, 99 (7) 4680-4685) have found significant up-regulation of several members of the apolipoprotein L family in the prefrontal cortex of schizophrenia brains. Middleton et al (Journal of Neuroscience, 2002, 22 (7) 2718-2729) have identified alterations of specific metabolic pathways in schizophrenia. However, the molecular basis of schizophrenia is only beginning to be understood. This has hampered development of effective treatments for schizophrenia, and reliable diagnosis of the disorder.

We have identified abnormalities in the expression levels of several genes in the prefrontal cortex of patients with schizophrenia compared with control samples. In particular, the expression level of the following genes was observed to be decreased in the prefrontal cortex of schizophrenia patients:

PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1;

Ornithine related genes: OAT; OAZIN; OAZ2;

Arginine related genes: ARG2;

ATP synthase (mitochondrial) genes: ATP6V1B2; ATP6IP2; ATP6V1C1;

ATP synthase (vacuolar) genes: ATP5J; ATP5G3; ATP5L; ATP5C1; ATP5F1; ATP5A1;

Complex 1 genes: NDUFA5; NDUFA6; NDUFAB1; NDUFB3; NDUFB6; NDUFB5; NDUFB1; NDUFS4; NDUFA4; NDUFC2; NDUFB4;

Complex 3 genes: UQCRH; UQCRFS1; UQCRC2; UQCRB; UQCRC2;

Complex 4 genes: COX7A2; COX7B; COX5A; COX17; COX11; COX7CP1; COX7BP1;

Holocytochrome c Synthetase genes: HCCS;

Adenine translocators genes: SLC25A4;

Voltage dependent anion channels (in mitochondrial outer-membrane) genes: VDAC2; VDAC1P; VDAC3;

Lactate metabolism genes: LDHB; LDHA;

Isocitrate dehydrogenase genes: IDH3B; IDH3A;

HMG related genes: HMGCR;

Glutamate metabolism genes: GLRX2.

The expression level of the following genes was observed to be increased in the prefrontal cortex of schizophrenia patients:

FBS1; WFS1; PRODH; AMT; CLN3; ACOX1; G6PD; GCDH; COL5A1; NY-REN-24; TXNL2; SOD3; BCAT2;

purine metabolism (matrix) genes: ALDH4A1; PYCR1;

metallo proteins genes: MT1X; MT1L; MT1G; MT1H; MT2A; MT1E; MT1F;

Arginine related genes: DDAH2;

Glycine/Serine metabolism genes: AMT;

HMG related genes: HMGCL;

Oxide related genes: EPHX1.

Table 1 gives the fold changes in expression of the above genes in the prefrontal cortex of schizophrenia brains compared with control samples, and includes Unigene, ReSeq, and Genbank details, and descriptions of the genes, including synonyms.

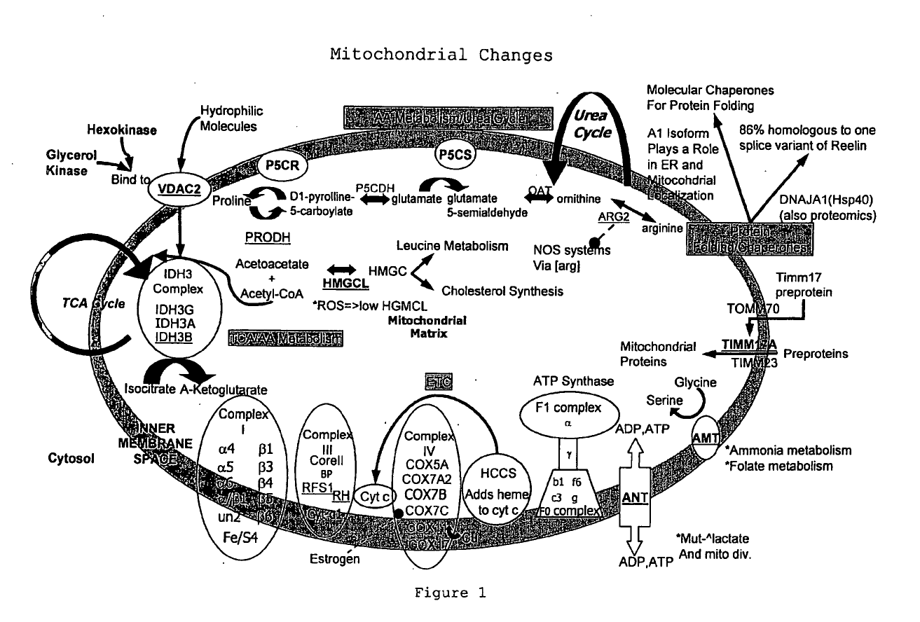

Many of the changes are mitochondrial changes. These are illustrated schematically in FIG. 1. The changes include changes in ROS stress systems (see the Example).

We have appreciated that these abnormalities can be used to identify potential therapeutic agents for the prevention, treatment, or amelioration of schizophrenia, for the diagnosis of schizophrenia or susceptibility to schizophrenia, to identify patients most likely to respond to a particular therapeutic treatment, and for selecting participants in clinical trials.

We have also carried out cluster analysis, filtering on oxidative stress and mitochondrial genes and found that 90% separation of schizophrenics from controls is achieved if expression of the following genes is downregulated: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; and expression of the following genes is upregulated: FBS1; WFS1; PRODH; AMT; CLN33; ACOX1; G6PD; GCDH; COL5A1; NY-REN-24; HMGCL; TXNL2; SOD3; BCAT2; MT1X.

Thus, the reliability of diagnosis of schizophrenia should be dramatically increased by determining the expression levels of the majority, preferably all, of these genes. Similarly, particularly effective screening methods are expected to be provided where the methods screen for compounds that cause appropriate changes in expression levels of the majority, preferably all, of these genes.

According to the invention there is provided a method for identifying a potential therapeutic agent for the prevention, treatment, or amelioration of schizophrenia, which comprises: contacting a cell with a candidate therapeutic agent, or administering a candidate therapeutic agent to an organism; determining whether expression of any of the following genes is altered in the cell or organism in response to the candidate therapeutic agent: PARG; OLR1; ARPC3; DNCLI1; PPM1A; ATPIF1; TIMM17A; DNAJA1; SST; NEUROD6; ICAP-1A; FLJ23251; KCNK1; FLJ3611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1; and identifying the candidate as a potential therapeutic agent if expression of one or more of the genes is altered.

Preferably it is determined whether expression of any of the genes is altered by comparing the expression level of the gene or genes in the presence and absence of the candidate therapeutic agent.

Preferably the candidate is identified as a potential therapeutic agent if expression of one or more of the following genes is increased: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; or expression of one or more of the following genes is decreased. FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MTG1; MT1H; MT2A; MT1E; MT1F; DDAH2; AMT; HMGCL; EPHX1.

More preferably the candidate is identified as a potential therapeutic agent if expression of the majority (preferably all) of the following genes is altered in response to the therapeutic agent: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; PRODH; AMT; CLN3; ACOX1; G6PD; GCDH; COL5A1; NY-REN-24; HMGCL; TXNL2; SOD3; BCAT2; MT1X.

In particular, the candidate is identified as a potential therapeutic agent if expression of the majority (preferably all) of the genes is altered in the following ways: an increase in expression of: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; a decrease in expression of: FBS1; WFS1; PRODH; AMT; CLN3; ACOX1; G6PD; GCDH; COL5A1; NY-REN-24; HMGCL; TXNL2; SOD3; BCAT2; MT1X.

The term “majority” used herein means more than 50%, preferably at least 60%, more preferably at least 70%, more preferably at least 80%, more preferably at least 90%, most preferably all.

Methods and assays of the invention may be implemented using any method suitable for measuring changes in gene expression. Methods of determining the expression level of a gene are well known to those of ordinary skill in the art. For example, this may be achieved by determining the level of mRNA or protein expressed from the gene. In particularly preferred embodiments, changes in expression level of a plurality of genes in response to the candidate therapeutic agent are determined using a microarray. Screening methods using microarrays for identifying candidate compounds for the treatment of neuropsychiatric disorders are described in detail in WO 03/042654. Use of microarrays is also discussed in detail below.

Any technique that is capable of measuring gene expression may be used. For instance, gene expression may also be measured in a preferred alternative embodiment by using a reverse transcription polymerase chain reaction (“RT-PCR”). Systems and kits for implementing such assays are commercially available from a number of suppliers, including Affymetrix (Santa Clara, Calif.), Agilent (Palo Alto, Calif.), Promega (Madison, Wis.), Xanthon (Research Triangle Park, N.C.), Illumina (San Diego, Calif.), Chromagen (San Diego, Calif.), Third Wave Technologies (Madison, Wis.), Aclara (Mountain View, Calif.), Beckton Dickinson & Co. (Franklin Lakes, N.J.) and Luminex (Austin, Tex.).

Other examples of suitable methods for determining the level of mRNA expression are quantitative PCR (in particular, real-time quantitative PCR) performed on cDNA produced by reverse transcription of the mRNA, and Northern blotting.

In a preferred method of determining the level of mRNA expressed, total RNA is obtained from the biological sample, cDNA is synthesized from mRNA of the gene, and the cDNA is used for real-time quantitative PCR analysis to determine the level of the mRNA in the sample.

Other examples of suitable methods for determining the level of protein expression are Western blotting and enzyme-linked immunosorbent assay (ELISA).

A binding partner of an expression product of the gene, may be used to detect the level of that expression product. The binding partner may be a protein, preferably an antibody or antibody fragment. The antibody or antibody fragment should bind specifically to the expression product so that the level of the expression product in the biological sample can be determined.

The binding partner may be a nucleic acid capable of hybridizing to a nucleic acid expression product of the gene, or to nucleic acid derived therefrom. The nucleic acid should hybridize specifically (for example under conditions of high stringency—see the section on Microarrays below) to the nucleic acid expression product, or nucleic acid derived therefrom, so that the level of the nucleic acid expression product in the biological sample can be determined. A preferred nucleic acid binding partner is an oligonucleotide primer for the synthesis of cDNA by reverse transcription from mRNA of the gene.

In some preferred embodiments, the level of a nucleic acid expression product of the gene is determined by amplification of that nucleic acid expression product, for example by PCR. Thus, primers capable of amplifying the nucleic acid expression product are provided. Nucleic acid capable of hybridizing (preferably under conditions of high stringency) to nucleic acid that is complementary to a nucleic acid expression product of the gene and/or nucleic acid which is a binding partner (preferably under conditions of high stringency) of an expression product of the gene may be used to amplify a nucleic acid expression product of the gene, for example to detect an expression product of the gene.

In preferred embodiments of the invention, screening for potential therapeutic agents is carried out by contacting a candidate therapeutic agent with cultured cells or cell lines.

Preferably, the cells are neuronal cells, or are cells that have an expression profile that is typical of neuronal cells or, alternatively, they may be cells that can be manipulated to produce an expression profile typical of neuronal cells. The cells or cell lines used will also, preferably, give rise to reproducible changes in their gene expression profiles when contacted with one or more known antipsychiatric drugs (for example, valproate, Haloperidol, Pirenzepine, Perazine, Risperdal, Famotidine, Zyprexa, Clozaril, Mesoridazine, Quetiapine, Risperidone, Olanzapine, or Clozapine). In particularly preferred embodiments, these changes will be opposite changes that are observed in schizophrenia. That is to say, in such embodiments, genes (or their homologs) normally expressed at higher levels in schizophrenia are preferably expressed at lower levels in cells or cell lines contacted with the known antipsychiatric drug, and vice-versa.

In a preferred embodiment, pluripotent neuronal stem cell lines are used in these aspects of the invention. Such cell lines are well known in the art, and methods to induce or enhance the differentiation of such stem cell lines have been described. For example, U.S. Provisional Patent Application Ser. Nos. 60/299,152 and 60/299,066 (both filed on Jun. 18, 2001) describe methods for inducing differentiation in neuronal stem cells by exposure to chemicals (for example, valproate and buspirone). In other embodiments, such cells may be differentiated, e.g., using antisense strategies and/or routine techniques of molecular biology to develop stable, transfected cell lines.

Alternatively, however, cells or cell lines may also be obtained from patients having a neuropsychiatric disorder, particularly schizophrenia, or from an animal model of schizophrenia.

In some preferred embodiments of the invention a human neuroblastoma cell line known as NBFL (Symes et al, Proc. Natl. Acad. Sci. U.S.A. 1993, 90(2):572-576) may be used. Suitable culture conditions for this cell line are described in WO 03/042654 (on page 53, lines 3-6).

It will be appreciated that cells or cell cultures used in the methods of this invention should be carefully controlled for parameters such as the cell passage number, cell density (e.g., in microplate wells), the method(s) by which cells are dispensed, and growth time after dispensing. It is also preferable to repeat mRNA and/or protein expression levels measured for a cell or cell line under particular conditions, to confirm that the measured levels are reproducible.

In other preferred embodiments of the invention, screening for potential therapeutic agents is carried out by administering a candidate therapeutic agent to an organism, preferably a non human animal (such as a mouse, rat, rabbit, monkey, guinea pig, dog or cat), although in some circumstances it may be desirable to administer a candidate therapeutic agent to a human, for example as part of a clinical trial. Preferably, the non human animal is an animal model of Schizophrenia (for example, phencyclidine treated rodents (Sams-Dodd Rev Neurosci (1999) 10, 59-90), an animal model of deficient sensorimotor gating (Swerdlow and Geyer Schizophr Bull (1998) 24:2 285-301), neonatal insult to the hippocampal region (Beauregard and Bachevalier Can J Psychiatry (1996) September 41:7 446-56), models based on neonatal excitotoxic hippocampal damage (Lillrank et al, Clin Neurosci (1995) 3:2 98-104), attention deficit models (Feldon et al, J Psychiatr Res 4. 345-66), NMDA deficient rodent models (Mohn et al, Cell (1999) 98, 427-436), animals that show decreased expression of mRNAs for synaptophysin, GAP43, cholecstokinin, and non-NMDA glutamate receptor subunits (GLU R1 and 2), particularly in CA 3-4 associated with Schizophrenia (Weinbrger Biol Psychiatry (1999) Feburary 5 45;:4 395-402, mice homozygous for PRODH2 deficiency (Gogos, J. A., et al, 1999, Nat. Genet. 21, 434-439).

It will be appreciated that where non human cells, organisms, or extracts are used in accordance with the invention, the proteins and genes specified will normally refer to the appropriate homologues of the human proteins or genes (i.e. the equivalent proteins and genes in that non human cell or organism) that are identified herein as being abnormally expressed in the prefrontal cortex of schizophrenia patients. In some embodiments (for example in some embodiments of the screening assays and methods described below), the human gene(s) or protein(s) may be recombinantly expressed in a non human organism, cell, system, or extract.

According to the invention there is also provided use of any of the following in a screening assay to identify a potential therapeutic agent for the prevention, treatment, or amelioration of schizophrenia: (i) proteins encoded by the following genes: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1; or

ii) nucleic acid encoding any of the proteins of (i) above. The nucleic acid may be RNA (for example mRNA, or DNA, including cDNA).

There is also provided according to the invention use of a regulator of expression of any of (i) above, in a screening assay to identify a potential therapeutic agent for the prevention, treatment, or amelioration of schizophrenia.

There is further provided according to the invention use of a binding partner of any of (i) or (ii) above in a screening assay to identify a potential therapeutic agent for the prevention, treatment, or amelioration of schizophrenia.

According to the invention there is also provided use of an expression vector comprising nucleic acid encoding any of (i) above in a screening assay to identify a potential therapeutic agent for the prevention, treatment, or amelioration of schizophrenia.

There is further provided according to the invention use of a cell or cell line expressing nucleic acid encoding any of (i) above in a screening assay to identify a potential therapeutic agent for the prevention, treatment, or amelioration of schizophrenia. Preferably the cell is a neural cell, or an oligodendrocyte.

There is also provided according to the invention a recombinant non-human animal in which expression of a gene encoding any of the proteins of (i) above is altered compared with expression of the corresponding gene in a normal animal. Preferably expression of two or more of the genes is altered. Expression of the gene or genes in the recombinant animal may be increased or decreased. Where expression is decreased, preferably the animal is a knockout animal for the gene or genes. Methods for providing recombinant animals are well known to those of skill in the art.

Preferably expression of PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2 is decreased in the recombinant animal.

Preferably expression of FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1 is increased in the recombinant animal.

Preferably the recombinant animal is a mouse. Other suitable non-human animals include rats, chickens, cows, monkeys, or rabbits.

The invention also provides use of a recombinant non-human animal of the invention as an animal model for schizophrenia.

According to the invention there is also provided use of a recombinant non-human animal of the invention, or cells obtained or derived from the animal, in a screening assay to identify a potential therapeutic agent for the prevention, treatment, or amelioration of schizophrenia.

Screening assays in accordance with the invention are described in more detail below:

A screening assay for identifying a potential therapeutic agent for the prevention, treatment, or amelioration of schizophrenia may comprise screening for a modulator of expression of a gene encoding any of the proteins of (i) above by: providing a system capable of expressing a gene or nucleic acid encoding any of the proteins of (i) above; maintaining the system under conditions for expression of the gene or nucleic acid in the presence and absence of a candidate modulator of expression of the gene; and determining the expression level of the gene or nucleic acid in the presence and absence of the candidate modulator.

The term “modulator” is used herein to mean an upregulator, or downregulator of expression of the gene.

The system may be an in vitro system capable of transcription of the gene and/or translation of mRNA encoding the protein coded by the gene. A preferred system is a cell, such as a cultured cell or cell line. Preferably, the cell is a neuronal cell, or a cell that has an expression profile that is typical of neuronal cell. Alternatively, the cell may be a cell that can be manipulated to produce an expression profile typical of neuronal cells.

A microarray may be used to determine the expression level of a plurality of the genes.

An upregulator of expression of any of the following is expected to provide a potential therapeutic agent for the prevention, treatment, or amelioration of schizophrenia: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIFIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

A downregulator of expression of any of the following is expected to provide a potential therapeutic agent for the prevention, treatment, or amelioration of schizophrenia: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

Preferably screening assays of the invention screen for upregulators of expression of the majority (preferably all) of the following: PARG; VDAC2; OLR1; ARPC3; UQCRFS1; DNCLI1; PPM1A; ATPIF1; GLRX2; TIMM17A IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1. Alternatively, or additionally, screening assays of the invention preferably screen for downregulators of expression of the majority (preferably all) of the following: FBS1; WFS1; PRODH; AMT; CLN3; ACOX1; G6PD; GCDH; COL5A1; NY-REN-24; HMGCL; TXNL2; SOD3; BCAT2; MT1X.

In one embodiment, agents that modulate (i.e. upregulate or downregulate) the expression of any of the proteins of (i) above are identified by contacting cells expressing the protein with a candidate compound or a control compound (e.g., phosphate buffered saline (PBS)) and determining the expression of the protein, or mRNA encoding the protein.

The level of expression of a selected protein, or mRNA encoding the protein in the presence of the candidate compound is compared to the level of expression of the protein or mRNA encoding the protein in the absence of the candidate compound (e.g., in the presence of a control compound). The candidate compound can then be identified as a modulator of the expression of the protein based on this comparison. For example, when expression of the protein or mRNA is significantly greater in the presence of the candidate compound than in its absence, the candidate compound is identified as an upregulator of expression of the protein or mRNA. Alternatively, when expression of the protein or mRNA is significantly less in the presence of the candidate compound than in its absence, the candidate compound is identified as a downregulator of expression of the protein or mRNA. The level of expression of the protein or the mRNA that encodes it can be determined by methods known to those of skill in the art. For example, mRNA expression can be assessed by Northern blot analysis or RT-PCR, and protein levels can be assessed by western blot analysis.

In preferred embodiments, test compounds that modulate expression of one or more of the proteins of (i) above are identified in non human animals (e.g. mice, rats, monkeys, rabbits, or guinea pigs), preferably non human animal models for schizophrenia (examples of non human animal models are given above). In accordance with such embodiments, a test compound or a control compound is administered to the animals, and the effect of the test compound on expression of one or more of the proteins is determined. A test compound that alters the expression of any of the proteins (or a plurality of the proteins) can be identified by comparing the level of the selected protein or proteins (or mRNA(s) encoding the same) in an animal or group of animals treated with a test compound with the level of the protein(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the level or expression of any of the proteins of (i) above (or a plurality of the proteins) are identified in human subjects, preferably those having schizophrenia and most preferably those having severe schizophrenia In accordance with this embodiment, a test compound or a control compound is administered to the human subject, and the effect of a test compound on expression of the protein(s) is determined, by analyzing the expression of the protein or the mRNA encoding the same in a biological sample (e.g., CSF, blood, serum, plasma, or urine). A test compound that alters the expression of the protein(s) can be identified by comparing the level of the protein or mRNA encoding the same in a subject or group of subjects treated with a control compound to that in a subject or group of subjects treated with a test compound. Alternatively, alterations in the expression of the protein(s) can be identified by comparing the level of the protein(s) or mRNA(s) encoding the same in a subject or group of subjects before and after the administration of a test compound. Techniques known to those of skill in the art can be used to obtain the biological sample and analyze the mRNA or protein expression.

A test compound that changes the level or expression of the protein(s) or mRNA(s) towards levels detected in control subjects (e.g., humans free from schizophrenia) is selected for further testing or therapeutic use.

An alternative screening assay for identifying a potential therapeutic agent for the prevention, treatment, or amelioration of schizophrenia may comprise screening for a regulator of the activity of any of the proteins of (i) above by: contacting the protein with a candidate regulator and determining the activity of the protein in the presence and absence of the candidate regulator. The regulator may be an enhancer or activator, or an inhibitor, of the activity of the protein.

An enhancer or activator of the activity of any of the following proteins may provide a potential therapeutic agent for the prevention, treatment, or amelioration of schizophrenia: PARG; OLR1; ARPC3; DNCLI1; PPM1A; ATPIF1; TIMM17A; DNAJA1; SST; NEUROD6; ICAP-1A; FUJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

An inhibitor of the activity of any of the following proteins may provide a potential therapeutic agent for the prevention, treatment, or amelioration of schizophrenia: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

Preferably screening methods of the invention screen for enhancers or activators of the activity of the majority (preferably all) of the following proteins: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1. Alternatively, or additionally, screening assays of the invention preferably screen for inhibitors of the activity of the majority (preferably all) of the following proteins: FBS1; WFS1; PRODH; AMT; CLN3; ACOX1; G6PD; GCDH; COL5A1; NY-REN-24; HMGCL; TXNL2; SOD3; BCAT2; MT1X.

In one embodiment, agents that regulate the activity of any of the proteins of (i) above are identified by contacting a preparation comprising the protein, or cells expressing the protein with a test compound, or a control compound, and determining the ability of the test compound to regulate (i.e. enhance or activate, or inhibit) the activity of the protein. The activity of the protein can be assessed by detecting induction of a cellular signal transduction pathway of the protein (e.g. intracellular Ca2+, diacylglycerol, IP3, etc.), detecting catalytic or enzymatic activity of the protein on a suitable substrate, detecting the induction of a reporter gene (e.g., a regulatory element that is responsive to the protein and is operably linked to a nucleic acid encoding a detectable marker, e.g. luciferase), or detecting a cellular response, for example, cellular differentiation, or cell proliferation. Techniques known to those of skill in the art can be used for measuring these activities (see, e.g., U.S. Pat. No. 5,401,639, which is incorporated herein by reference). The candidate compound can then be identified as a regulator of the activity of the protein by comparing the effects of the candidate compound to the control compound. Suitable control compounds include phosphate buffered saline (PBS) and normal saline (NS).

In another embodiment, test compounds that regulate the activity of any of the proteins of (i) above (or their homologue) or a biologically active portion thereof are identified in non-human animals (e.g., mice, rats, monkeys, rabbits, and guinea pigs), preferably non-human animal models for schizophrenia (examples of non-human animal models are given above). In accordance with this embodiment, a test compound or a control compound is administered to the animals, and the effect of a test compound on the activity of protein(s) is determined. A test compound that alters the activity of the protein (or a plurality of the proteins) can be identified by assaying animals treated with a control compound and animals treated with the test compound. The activity of the protein can be assessed by detecting induction of a cellular second messenger of the protein (e.g., intacellular Ca2+, diacylglycerol, IP3, etc.), detecting catalytic or enzymatic activity of the protein or binding partner thereof, detecting the induction of a reporter gene (e.g., a regulatory element that is responsive to the protein operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of the protein (see, e.g., U.S. Pat. No. 5,401,639, which is incorporated herein by reference).

In another embodiment, test compounds that regulate the activity of any of the proteins of (i) above (or a plurality of the proteins) are identified in human subjects, preferably those having schizophrenia and most preferably those with severe schizophrenia. In this embodiment, a test compound or a control compound is administered to the human subject, and the effect of a test compound on the activity of the protein(s) is determined. A test compound that alters the activity of the protein(s) can be identified by comparing biological samples from subjects treated with a control compound to samples from subjects treated with the test compound. Alternatively, alterations in the activity of the protein(s) can be identified by comparing the activity of the protein(s) in a subject or group of subjects before and after the administration of a test compound. The activity of the protein(s) can be assessed by detecting in a biological sample (e.g., CSF, serum, plasma, or urine) induction of a cellular signal transduction pathway of the protein (e.g., intracellular Ca2+, diacylglycerol, IP3, etc.), catalytic or enzymatic activity of the protein or a binding partner thereof, or a cellular response, for example, cellular differentiation, or cell proliferation. Techniques known to those of skill in the art can be used to detect changes in the induction of a second messenger of the protein(s) or changes in a cellular response. For example, RT-PCR can be used to detect changes in the induction of a cellular second messenger.

A test compound that changes the activity of the protein(s) towards the activity found in control subjects (e.g., humans free from schizophrenia) is selected for further testing or therapeutic use.

A further screening assay for identifying a potential therapeutic agent for the prevention, treatment, or amelioration of schizophrenia may comprise screening for a regulator of the interaction of any of the proteins of (i) above with a binding partner required for the biological effect of the protein by: contacting the protein with the binding partner in the presence of a candidate regulator, and determining binding of the protein to its binding partner in the presence and absence of the candidate regulator. The regulator may be an enhancer or activator, or an inhibitor, of the interaction of the protein with the binding partner.

An enhancer or activator of the interaction of any of the following proteins with a binding partner required for the biological effect of the protein may provide a potential therapeutic agent for the prevention, treatment, or amelioration of schizophrenia: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

An inhibitor of the interaction of any of the following proteins with a binding partner required for the biological effect of the protein may provide a potential therapeutic agent for the prevention, treatment, or amelioration of schizophrenia: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

Preferably screening assays of the invention screen for enhancers or activators of the interaction of the majority (preferably all) of the following proteins with binding partners required for the biological effects of the proteins: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1. Alternatively, or additionally, screening assays of the invention preferably screen for inhibitors of the interaction of the majority (preferably all) of the following proteins with binding partners required for the biological effects of the proteins: FBS1; WFS1; PRODH; AMT; CLN3; ACOX1; G6PD; GCDH; COL5A1; NY-REN-24; HMGCL; TXNL2; SOD3; BCAT2; MT1X.

In one embodiment, a cell-based assay system is used to identify candidates that regulate the activity of any of the proteins of (i) above. In a primary screen, a plurality (e.g., a library) of compounds are contacted with cells that naturally or recombinantly express: (i) any of the proteins of (i) above; and (ii) a protein that is responsible for processing of the protein in order to identify compounds that modulate the production, degradation, or post-translational modification of the protein. If desired, compounds identified in the primary screen can then be assayed in a secondary screen against cells naturally or recombinantly expressing the specific protein of interest. The ability of the candidate compound to modulate the production, degradation or post-translational modification of the protein can be determined by methods known to those of skill in the art, including without limitation, flow cytometry, a scintillation assay, immunoprecipitation and western blot analysis.

In another embodiment, agents that competitively bind to any of the proteins of (i) above are identified in a competitive binding assay. In accordance with this embodiment, cells expressing the protein are contacted with a candidate compound and a compound known to interact with the protein. The ability of the candidate compound to competitively bind to the protein is then determined. Alternatively, agents that competitively bind to any of the proteins of (i) above are identified in a cell-free assay system by contacting the protein with a candidate compound and a compound known to interact with the protein. As stated above, the ability of the candidate compound to interact with the protein can be determined by methods known to those of skill in the art. These assays, whether cell-based or cell-free, can be used to screen a plurality (e.g., a library) of candidate compounds.

A further screening assay for identifying a potential therapeutic agent for the prevention, treatment, or amelioration of schizophrenia may comprise screening for a binding partner of any of the proteins of (i) above by: contacting the protein with a sample comprising a candidate binding partner, and determining whether the candidate binding partner binds to the protein.

Preferably screening assays of the invention screen for binding partners of the majority (preferably all) of the following proteins: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; FBS1; WFS1; PRODH; AMT; CLN3; ACOX1; G6PD; GCDH; COL5A1; NY-REN-24; HMGCL; TXNL2; SOD3; BCAT2; MT1X.

In one embodiment, candidates that interact with the protein are identified in a cell-based assay system. In accordance with this embodiment, cells expressing the protein are contacted with a candidate compound or a control compound and the ability of the candidate compound to interact with the protein is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the protein endogenously or be genetically engineered to express the protein. In certain instances, the protein or the candidate compound is labelled, for example with a radioactive label (such as ³²P, ³⁵S) or a fluorescent label (such as fluorescein isothiocyanate, rhodamine, phycoertrin, phycocyanin, allophycocyanin, o-phthaldehyde or fluorescamine) to enable detection of an interaction between the protein and a candidate compound. The ability of the candidate compound to interact directly or indirectly with the protein can be determined by methods known to those of skill in the art. For example, the interaction between a candidate compound and the protein can be determined by flow cytometry, a scintillation assay, immunoprecipitation or western blot analysis.

In another embodiment, agents that bind to any of the proteins of (i) above are identified in a cell-free assay system. In accordance with this embodiment, a native or recombinant protein is contacted with a candidate compound or a control compound and the ability of the candidate compound to interact with the protein is determined. If desired, this assay may be used to screen a plurality (e.g. a library) of candidate compounds. Preferably, the protein is first immobilized, by, for example, contacting the protein with an immobilized antibody which specifically recognizes and binds to it, or by contacting a purified preparation of the protein with a surface designed to bind proteins. The protein may be partially or completely purified (e.g., partially or completely free of other polypeptides) or part of a cell lysate. Further, the protein may be fused to another protein domain, such as glutathionine-S-transferase, as part of a fusion protein. Alternatively, the protein can be biotinylated using techniques well known to those of skill in the art (e.g. biotinylation kit, Pierce Chemicals; Rockford, Ill.). The ability of the candidate compound to interact with the protein or fusion protein can be can be determined by methods known to those of skill in the art.

In yet another embodiment, any of the proteins of (i) above is used as a “bait protein” in a two-hybrid assay or three-hybrid assay to identify other proteins that bind to the protein (see, e.g., U.S. Pat. No. 5,283,317; Zervos et al, Cell (1993) 72:223-232; Madura et al, J. Biol. Chem. (1993) 268:12046-12054; Bartel et al, Bio/Techniques (1993) 14:920-924; Iwabuchi et al, Oncogene (1993) 8:1693-1696; and PCT Publication No. WO 94/10300).

Examples of potential therapeutic agents, candidate modulators, candidate regulators, or candidate binding partners include, but are not limited to, nucleic acids (e.g., DNA and RNA), carbohydrates, lipids, proteins, peptides, peptidomimetics, small molecules and other drugs. Candidates can be obtained using any of the numerous approaches in combinatorial library methods known in the art, including: biological libraries; spatially addressable parallel solid phase or solution phase libraries; synthetic library methods requiring deconvolution; the “one-bead, one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. (1997) 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al, Proc. Natl. Acad. Sci. USA (1993) 90:6909; Erb et al, Proc. Natl. Acad. Sci. USA (1994) 91:11422; Zuckermann et a, J. Med. Chem. (1994) 37:2678; Cho et al, Science (1993) 261:1303; Carrell et al, Angew. Chem. Int. Ed. Engl. (1994) 33:2059; Carell et al, Angew. Chen Int. Ed. Engl. (1994) 33:2061; and Gallop et al, J. Med. Chem.

(1994) 37:1233; each of which is incorporated herein in its entirety by reference.

Libraries of compounds may be presented, e.g., presented in solution (e.g., Houghten, Bio/Techniques (1992) 13:412-421), or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al, Proc. Natl. Acad. Sci. USA (1992) 89:1865-1869) or phage (Scott and Smith, Science (1990) 249:386-390; Devlin, 1 5 Science (1990) 249:404-406; Cwirla et al, Proc. Natl. Acad. Sci. USA (1990) 87:6378-6382; and Felici, J. Mol. Biol. (1990) 222:301-310), each of which is incorporated herein in its entirety by reference.

There is also provided according to the invention a method of diagnosing whether a subject has, or is at risk of developing schizophrenia, which comprises determining the level of any of the proteins of (i) above, or the expression level of a gene encoding any of the proteins of (i) above, in a biological sample obtained from the subject, or in a sample derived from a biological sample obtained from the subject.

Preferably the expression level of the majority (preferably all) of the following genes, or the levels of the majority (preferably all) of the proteins encoded by the following genes is determined in a biological sample obtained from the subject, or in a sample derived from a biological sample obtained from the subject: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; PRODH; AMT; CLN3; ACOX1; G6PD; GCDH; COL5A1; NY-REN-24; HMGCL; TXNL2; SOD3; BCAT2; MT1X.

If the level of the proteins or expression products in the brain is abnormal (for example compared with control samples from non schizophrenic brains), the subject is diagnosed as either having schizophrenia, or being at risk of developing schizophrenia.

In particular, the subject is diagnosed as either having schizophrenia, or being at risk of developing schizophrenia, if the expression level of the majority (preferably all) of the following genes, or the level of the majority (preferably all) of the proteins encoded by the following genes is reduced compared to a normal subject: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; and the expression level of the majority (preferably all) of the following genes, or the level of the majority (preferably all) of the proteins encoded by the following genes is increased compared to a normal subject: FBS1; WFS1; PRODH; AMT; CLN3; ACOX1; G6PD; GCDH; COL5A1; NY-REN-24; HMGCL; TXNL2; SOD3; BCAT2; MT1X.

It is expected that upto 90% reliability of diagnosis of schizophrenia can be achieved by such methods.

The biological sample may comprise any of the following: CNS tissue, brain tissue, cells isolated from the prefrontal cortex, cells isolated from the developing neuroepithelium; a neural stem cell; a progenitor cell; cerebrospinal fluid (CSF).

Brain Tissue Samples: in certain embodiments, brain cells and tissues for use in methods of the invention may be obtained from individuals (e.g., from patients) in a biopsy. However, brain surgeries permitting a biopsy are relatively rare and primarily involve surgical excisions (e.g., for the treatment of epilepsy) rather than brain regions relevant to neuropsychiatric disorders such as schizophrenia. In certain embodiments, however, useful profiles may be obtained from cultured peripheral nervous system neurons, such as rhinoneuroepithelial cells. Such cells may be readily obtained from a nasal biopsy.

The term “cerebrospinal fluid” (CSF) used herein refers to the fluid that surrounds the bulk of the central nervous system, as described in Physiological Basis of Medical Practice (J. B. West, ed., Williams and Wilkins, Baltimore, Md. 1985). CSF includes ventricular CSF and lumbar CSF.

Cells isolated from the developing human neuroepithelium can be isolated in culture and grown as aggregates termed neurospheres (Svendsen C N, and Smith A G, Trends Neurosci 1999 August; 22(8): 357-64). These contain a mixture of neural stem and progenitor cells, can be propagated in culture for extended time periods, and hold potential as a source of tissue for repairing the damaged CNS. According to the invention, the sample derived from the biological sample may be a neurosphere.

CSF may be analysed by two-dimensional electrophoresis to determine the level of one or more of the proteins of (i) above. This technique is well known to those of skill in the art. Methods of diagnosing schizophrenia using two-dimensional electrophoresis are described in detail in WO 01/63293.

Preferably the biological sample comprises peripheral tissue or a peripheral cell type in which the level of the protein, or the expression level of the gene, correlates with the level of the corresponding protein, or the expression level of the corresponding gene, in the prefrontal cortex.

Suitable peripheral tissue may comprise blood (consisting of plasma and blood cells), serum, plasma, urine, or liver or spleen cells. It is possible that a correlated level of protein, or correlated gene expression, may occur in one or more types of blood cell but not in others. In this case, it may be necessary to use blood cells of that type, or those types, which have been separated at least from some of the types of blood cells that do not have correlated levels or correlated expression. If a correlated level of protein, or correlated gene expression, occurs in more than one type of blood cell, blood cells of each type could be separated and, if necessary, pooled together for the determination.

A correlated level of protein, or correlated gene expression may occur in erythrocytes (red cells), platelets, or leukocytes (granulocytes: neutrophils, eosinophils, or basophils; or lymphoid cells: lymphocytes or monocytes).

A microarray may be used in a method of diagnosis of the invention to determine the level of a plurality of proteins, or the expression level of a plurality of genes.

According to the invention there is provided a microarray which is a gene chip for use in a method of diagnosis of the invention, the gene chip comprising a plurality of different probes capable of hybridising to nucleic acid expression products, or nucleic acid derived from nucleic acid expression products, of the majority (preferably all) of the following genes: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FUJ23251; KCNK1; SLC25A4; FLJ13611; HIRIP5; COX7A2; COX5A; TAC1; UQCRH; MAGEH1; C13orf12; EBNA1BP2; DIRAS2; MPPE1; FBS1; WFS1; PRODH; AMT; CLN3; ACOX1; G6PD; GCDH; COL5A1; NY-REN-24; HMGCL; TXNL2; SOD3; BCAT2; MT1X.

There is also provided according to the invention a kit for the diagnosis of schizophrenia that comprises a means for detecting the protein or expression product of a gene encoding the protein, or of nucleic acid derived from a nucleic acid expression product of the gene. The detecting means may comprise a binding partner of the protein (such as an antibody), or a binding partner of nucleic acid encoding the protein, and/or a nucleic acid capable of hybridizing to nucleic acid that is complementary to a nucleic acid expression product of the gene.

Kits of the invention may allow for the detection of expression products of a plurality of the genes, or of nucleic acids derived from nucleic acid expression products of a plurality of the genes.

Preferred kits comprise means for detecting the protein or expression products (or nucleic acid derived from the nucleic acid expression products) of the majority (preferably all) of the following genes: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FIJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; PRODH; AMT; CLN3; ACOX1; G6PD; GCDH; COL5A1; NY-REN-24; HMGCL; TXNL2; SOD3; BCAT2; MT1X.

Each detecting means may comprise a binding partner of the protein and/or a nucleic acid (or analogue) capable of hybridizing to nucleic acid that is complementary to a nucleic acid expression product of the gene. Each detecting means may comprise a binding partner of a nucleic acid expression product of the gene. According to a preferred embodiment, the expression levels may be determined using a microarray, such as a gene chip (see below).

There is also provided according to the invention a kit comprising a pair of primers (each of which is preferably 6-30 nucleotides, more preferably 10-30 nucleotides, most preferably 10-20 nucleotides) that under appropriate reaction conditions can prime amplification of at least a portion of a nucleic acid expression product of any of the genes encoding the proteins of (i) above, or of nucleic acid derived from the nucleic acid expression product. The amplification may be, for example, by polymerase chain reaction (see, for example, Innis et al, 1990, PCR Protocols, Academic Press, Inc., San Diego, Calif.), ligase chain reaction (see EP 320, 308), use of Qβ replicase, cyclic probe reaction, or other methods known in the art.

Kits of the invention may optionally further comprise one or more of the following:
i) instructions for using the detecting means for diagnosis, prognosis, or therapeutic monitoring;
ii) a labelled moiety for detecting the detecting means;
iii) a solid phase to which the detecting means is immobilised;
iv) a predetermined amount of an isolated expression product of one or more of the genes for use as a standard, or control;
v) a label or insert indicating regulatory approval for diagnostic, prognostic or therapeutic use as appropriate.

If no labelled moiety is provided, the detecting means itself may be labelled with a detectable label (for example, a chemiluminescent, enzymatic, fluorescent, or radioactive label).

There is also provided according to the invention a method of diagnosing whether a subject has, or is at risk of developing schizophrenia, which comprises determining the level of any of the proteins of (i), or the expression level of a gene encoding any of the proteins of (i) above, in the brain (preferably the prefrontal cortex) of the subject.

The level of more than one of the proteins of (i) above, or the expression level of more than one of the genes encoding the proteins of (i) above may be determined. This may increase the accuracy of the diagnosis.

Preferably the expression level of the majority (preferably all) of the following genes, or the levels of the majority (preferably all) of the proteins encoded by the following genes in the brain (preferably the prefrontal cortex) of the subject is determined: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; PRODH; AMT; CLN3; ACOX1; G6PD; GCDH; COL5A1; NY-REN-24; HMGCL; TXNL2; SOD3; BCAT2; MT1X.

If the level of the protein or expression product in the brain is abnormal, the subject is diagnosed as either having schizophrenia, or being at risk of developing schizophrenia.

In particular, the subject is diagnosed as either having schizophrenia, or being at risk of developing schizophrenia, if the level of any of the following proteins, or the expression level of a gene encoding any of the following proteins is reduced compared to a normal subject: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

The subject is diagnosed as either having schizophrenia, or being at risk of developing schizophrenia, if the level of any of the following proteins, or the expression level of a gene encoding any of the following proteins is increased compared to a normal subject: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

There is further provided according to the invention a method of prevention, treatment, or amelioration of schizophrenia which comprises increasing the level or activity of any of the following proteins in the brain (in particular the prefrontal cortex) of a subject in need of such prevention, treatment, or amelioration: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

There is also provided according to the invention a method of prevention, treatment, or amelioration of schizophrenia which comprises reducing the level or activity of any of the following proteins in the brain (in particular the prefrontal cortex) of a subject in need of such prevention, treatment, or amelioration: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

There is further provided according to the invention a method of prevention, treatment, or amelioration of schizophrenia which comprises increasing the level or activity of the majority (preferably all) of the following proteins in the brain (in particular the prefrontal cortex) of a subject in need of such prevention, treatment, or amelioration: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; and reducing the level or activity of the majority (preferably all) of the following proteins in the brain (in particular the prefrontal cortex) of the subject: FBS1; WFS1; PRODH; AMT; CLN3; ACOX1; G6PD; GCDH; COL5A1; NY-REN-24; HMGCL; TXNL2; SOD3; BCAT2; MT1X.

The level of a protein may be altered by gene therapy. Use of gene therapy in relation to the treatment of schizophrenia is described in detail in WO 01/63293 at Section 5.14.2, pages 108-112. The content of this section is incorporated herein by reference in its entirety.

The level of a protein may be altered by use of a regulator of expression of a gene coding for the protein. The level or activity of a protein may be increased by administering the protein, or a fragment thereof, or nucleic acid encoding the protein or fragment to the subject. The activity of a protein may be regulated by administering an agent known to regulate activity of the protein. The level or activity of a protein may be decreased by administering an antisense olignoucleotide (use of antisense nucleic acids in relation to the treatment of schizophrenia is discussed in detail in WO 01/63293, Section 5.14.4-6, pages 113-116), a ribozyme (use of ribozymes in relation to the treatment of shcizophrenia is discussed in detail in WO 01/63293, Section 5.14.7, pages 116-119), a short interfering (si) RNA (siRNAs are reviewed in Dykxhoorn et al, 2003, Nature Reviews, Molecular Cell Biology, Vol. 4, 457), an antibody directed against the protein, or a compound that inhibits the activity of the protein.

In preferred embodiments, therapy or prophylaxis is tailored to the needs of an individual patient known to have, or suspected of having schizophrenia. According to such embodiments, it is determined which of the proteins of (i) above are present in abnormal levels (i.e. more or less than normal subjects), or which of the genes encoding the proteins are expressed at abnormal levels, and the patient is administered with compounds that promote or reduce the level or activity of the proteins or the level of expression of the genes as appropriate.

Thus, according to a further aspect of the invention there is provided a method for identifying a schizophrenia patient, or a patient suspected of having schizophrenia, who is likely to respond to a therapeutic treatment that alters the level or activity of any of the proteins of (i) above, which comprises: determining the level of expression of any of the genes encoding the proteins of (i) above in a patient, or in a biological sample obtained from the patient; and identifying the patient as being likely to respond to the therapeutic treatment if the level of expression of the gene or genes is altered compared to a normal subject.

In particular, the level of expression of any of the genes encoding the following proteins in the patient should be decreased, and the therapeutic treatment should be one that increases the level or activity of any of the following proteins: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATPSF1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2. Alternatively, or additionally, the level of expression of any of the genes encoding the following proteins in the patient should be increased, and the therapeutic treatment should be one that decreases the level or activity of any of the following proteins: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

Preferably the level of expression of the majority (preferably all) of the genes encoding the following proteins in the patient should be decreased, and the therapeutic treatment should be one that increases the level or activity of the majority (preferably all) of the following proteins: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1. Alternatively or additionally the level of expression of the majority (preferably all) of the genes encoding the following proteins in the patient should be increased, and the therapeutic treatment should be one that decreases the level or activity of the majority (preferably all) of the following proteins: FBS1; WFS1; PRODH; AMT; CLN3; ACOX1; G6PD; GCDH; COL5A1; NY-REN-24; HMGCL; TXNL2; SOD3; BCAT2; MT1X.

According to a further aspect of the invention, there is provided a method for selecting a participant in a clinical trial to determine the effectiveness of a potential therapeutic agent for the prevention, treatment, or amelioration of schizophrenia, which comprises: determining the level of expression of any of the genes encoding the proteins of (i) above in a candidate participant, or in a biological sample obtained from the candidate participant; and selecting the candidate for the clinical trial if the level of expression of the gene or genes is altered compared to a normal subject.

In particular, the candidate is selected for the clinical trial if the level of expression of any of the genes encoding the following proteins is decreased: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2. Alternatively, or additionally, the candidate is selected for the clinical trial if the level of expression of any of the genes encoding the following proteins is increased: FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

Preferably the candidate is selected for the clinical trial if the level of expression of the majority (preferably all) of the genes encoding the following proteins is decreased: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1. Alternatively, or additionally, the candidate is selected for the clinical trial if the level of expression of the majority (preferably all) of the genes encoding the following proteins is increased: FBS1; WFS1; PRODH; AMT; CLN3; ACOX1; G6PD; GCDH; COL5A1; NY-REN-24; HMGCL; TXNL2; SOD3; BCAT2; MT1X.

Microarrays

The term “microarray” is used herein to refer to any ordered arrangement (e.g., on a surface or substrate) of different molecules, referred to herein as “probes”. Each different probe of a microarray specifically recognizes and/or binds to a particular molecule, which is referred to herein as its “target”. Microarrays are therefore useful for simultaneously detecting the presence or absence of a plurality of different target molecules, e.g., in a sample. In preferred embodiments, microarrays used in the present invention are “addressable microarrays” where each different probe is associated with a particular “address”. For example, in preferred embodiments where the probes are immobilized on a surface or a substrate, each different probe of the addressable microarray may be immobilized at a particular, known location on the surface or substrate. The presence or absence of that probe's target molecule in a sample may therefore be readily determined by simply determining whether a target has bound to that particular location on the surface or substrate.

In some embodiments of the invention, a microarray may comprise a plurality of different antibodies that each bind to a particular target protein or antigen. More preferably, however, the methods of the invention are practiced using nucleic acid microarrays that comprise a plurality of nucleic acid probes immobilized on a surface or substrate. The different nucleic acid probes are complementary to, and therefore hybridize, to different target nucleic acid molecules, e.g., in a sample. Thus such probes may be used to simultaneously detect the presence and/or abundance of a plurality of different nucleic acid molecules in a sample, including the expression of a plurality of different genes; e.g., the presence and/or abundance of different mRNA molecules, or of nucleic acid molecules derived therefrom (for example, cDNA or cRNA).

Preferably, nucleic acid molecules in the present invention are detected by hybridization to probes of a microarray. Hybridization and wash conditions are therefore preferably chosen so that the probe “specifically binds” or “specifically hybridizes” to a specific target nucleic acid. In other words, the nucleic acid probe preferably hybridizes, duplexes or binds to a target nucleic acid molecule having a complementary nucleotide sequence, but does not hybridize to a nucleic acid molecule having a non-complementary sequence. As used herein, one nucleotide sequence is considered complementary to another when, if the shorter of the polynucleotides is less than or equal to about 25 bases, there are no mismatches using standard base-pairing rules. If the shorter of the two nucleotides is longer than about 25 bases, there is preferably no more than a 5% mismatch. Preferably, the two nucleotides are perfectly complementary (i.e., no mismatches). In can be easily demonstrated that particular hybridization conditions are suitable for specific hybridization by carrying out the assay using negative controls. See, for example, Shalon et al., Genome Research 1996, 639-645; and Chee et at, Science 1996, 274:610-614.

Optimal hybridization conditions for use with microarrays will depend on the length (e.g., oligonucleotide versus ploynucleotide greater than about 200 bases) and type (e.g., RNA, DNA, PNA, etc.) of probe and target nucleic acid. General parameters for specific (i.e., stringent) hybridization conditions are as follows: low stringency hybridization conditions—5×SSC, 0.1% SDS, and no formamide; or 30% formamide, 5×SSC, 0.5% SDS; moderate stringency hybridization conditions—40% formamide, with 5× or 6×SCC; high stringency hybridization conditions—50% formamide, 5× or 6×SCC. SCC is a buffer solution commonly used for nucleic acid hybridizations and comprises 0.15 M NaCl, 0.015 M Na-citrate.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA.

For hybrids of greater than 100 nucleotides in length, equations for calculating Tm have been derived (see Sambrook et al., Molecular Cloning—A Laboratory Manual (2nd Ed.), Vol. 1-3, Cold Spring Harbor Laboratory, Cold Spring Harbor, N.Y., 1989). For hybridization with shorter nucleic acids, i.e., oligonucleotides, the position of mismatches becomes more important, and the length of the oligonucleotide determines its specificity (see Sambrook et al., supra). A minimum length for a hybridizable nucleic acid is at least about 10 nucleotides; preferably at least about 15 nucleotides; and more preferably the length is at least about 20 nucleotides.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature.

Because the melting temperature of oligonucleotides will depend on the length of the oligonucleotide sequences involved, suitable hybridization temperatures will vary depending upon the oligonucleotide molecules used. Exemplary temperatures may be 370C (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

For cDNA microarrays, such as those described by Schena et al. (Proc. Natl. Acad. Sci. USA 1996, 93:10614), typical hybridization conditions comprise hybridizing in 5×SSC and 0.2% SDS at 65° C. for about four hours, followed by washes at 25° C. in a low stringency wash buffer (for example, 1×SSC and 0.2% SDS), and about 10 minutes washing at 25° C. in a high stringency wash buffer (for example, 0.1×SSC and 0.2% SDS). Useful hybridization conditions are also provided, e.g., in Tijessen, Hybridization with Nucleic Acid Probes, Elsevier Sciences Publishers (1996), and Kricka, Nonisotopic DNA Probe Techniques, Academic Press, San Diego Calif. (1992).

In preferred embodiments of the invention, transcript microarrays are used to compare the steady state level of mRNAs between two cells, such as a first cell that has been exposed to a candidate therapeutic agent and a second cell that has not. In one embodiment, transcript microarrays are produced by hybridizing detectably labeled polynucleotides representing the mRNA transcripts present in a cell (e.g., fluorescently labeled cDNA synthesized from total cell mRNA) to a microarray.

Microarrays share certain characteristics. The arrays are preferably reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, and a gene for which the encoded mRNA is prevalent will have a relatively strong signal.

In preferred embodiments, cDNAs from two different cells, e.g., a cell exposed to a test compound and a cell of the same type not exposed to the compound, are hybridized to the binding sites of the microarray. The cDNA derived from each of the two cell types are differently labeled so that they can be distinguished. In one embodiment, for example, cDNA from a cell treated with a drug is synthesized using a fluorescein-labeled dNTP, and cDNA from a second cell not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the treated cell will fluoresce green when the fluorophore is stimulated and the cDNA from the untreated cell will fluoresce red. As a result, when the compound has no effect, either directly or indirectly, on the relative abundance of a particular mRNA in a cell, the mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the cell is exposed to a compound that, directly or indirectly, increases the prevalence of the mRNA in the cell, the ratio of green to red fluorescence will increase. When the drug decreases the mRNA prevalence, the ratio will decrease.

The use of a two-colour fluorescence labeling and detection scheme to define alterations in gene expression has been described, e.g., in Shena et al., Science 1995, 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a treated and untreated cell.

Nucleic acid microarrays are known in the art and preferably comprise a surface to which probes that correspond in sequence to gene products (e.g., cDNAs, mRNAs, cRNAs, polypeptides, and fragments thereof), can be specifically hybridized or bound at a known position. In one embodiment, the microarray is an array in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA, or a gene fragment.

Preferably, the microarray has binding sites for mRNA, or for nucleic acid derived from mRNA, expressed from the following genes: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; PRODH; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

More preferably the binding sites are for mRNA, or for nucleic acid derived from mRNA, expressed from the majority, preferably all, of the following genes: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; FBS1; WFS1; PRODH; AMT; CLN3; ACOX1; G6PD; GCDH; COL5A1; NY-REN-24; HMGCL; TXNL2; SOD3; BCAT2; MT1.

Preparing Nucleic Acids for Microarrays. As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length PCR methods are well known and are described, for example, in Innis et al., eds., 1990, PCR Protocols: A Guide to Methods and Applications, Academic Press Inc. San Diego, Calif.

An alternative means for generating the nucleic acid for the microarray is by synthesis of synthetic polynucleotides or oligonucleotides, e.g., using N-phosphonate or phosphoramidite chemistries (Froehler et al., Nucleic Acid Res. 1986, 14:5399-5407; McBride et al., Tetrahedron Lett. 1983, 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, for example, Egholm. et al., Nature 1993, 365:566-568. See, also, U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., Genomics 1995, 29:207-209). In yet another embodiment, the polynucleotide of the binding sites is RNA.

Attaching Nucleic Acids to the Solid Surface. The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena. et al., Science 1995, 270:467470. This method is especially useful for preparing microarrays of cDNA. See also DeRisi et al., Nature Genetics 1996, 14:457-460; Shalon et al., Genome Res. 1996, 6:639-645; and Schena et al., Proc. Natl. Acad. Sci. USA 1995, 93:10539-11286.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., Science 1991, 251:767-773; Pease et al., Proc. Natl. Acad. Sci. USA 1994, 91:5022-5026; Lockhart et al., Nature Biotech. 1996, 14:1675. See, also, U.S. Pat. Nos. 5,578,832; 5,556,752; and 5,510,270) or other methods for rapid synthesis and deposition of defined oligonucleotides (Blanchard et al., Biosensors & Bioelectronics 1996, 11:687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, Nuc. Acids Res. 1992, 20:1679-1684), may also be used In principal, any type of array, for example, dot blots on a nylon hybridization membrane (see, Sambrook et al., Molecular Cloning-A Laboratory Manual (2nd Ed.), Vol. 1-3, Cold Spring Harbor Laboratory, Cold Spring Harbor, N.Y., 1989), could be used, although, as will be recognized by those of skill in the art, very small arrays will be preferred because hybridization volumes will be smaller.

Generating Labeled Probes. Methods for preparing total and poly(A)+ RNA are well known and are described generally in Sambrook et al., supra. In one embodiment, RNA is extracted from cells of the various types of interest in this invention using guanidinium thiocyanate lysis followed by CsCl centrifugation (Chirgwin et al., Biochemistry 1979, 18:5294-5299). Poly(A)+ RNA is selected by selection with oligo-dT cellulose (see Sambrook et al., supra). Cells of interest may include, but are not limited to, wild-type cells, drug-exposed wild-type cells, modified cells, and drug-exposed modified cells.

Labeled cDNA is prepared from mRNA by oligo dT-primed or random-primed reverse transcription, both of which are well known in the art (see, for example, Klug & Berger, Methods Enzymol. 1987, 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled NTPs (Lockhart et al., Nature Biotech. 1996, 14:1675). In alternative embodiments, the cDNA or RNA probe can be synthesized in the absence of detectable label and may be labeled subsequently, e.g., by incorporating biotinylated dNTPs or NTP, or some similar means (e.g., photo-cross-linking a psoralen derivative of biotin to RNAs), followed by addition of labeled streptavidin (e.g., phycoerythrinconjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., Gene 1995, 156:207; Pietu et al., Genome Res. 1996, 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™ II, LTI Inc.) at 42° C. for 60 min.

Hybridization to Microarrays. Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a noncomplementary nucleic acid sequence. As used herein, one polynucleotide sequence is considered complementary to another when, if the shorter of the polymicleotides is less than or equal to 25 bases, there are no mismatches using standard base-pairing rules or, if the shorter of the polynucleotides is longer than 25 bases, there is no more than a 5% mismatch Preferably, the polynucleotides are perfectly complementary (no mismatches). It can easily be demonstrated that specific hybridization conditions result in specific hybridization by carrying out a hybridization assay including negative controls (see, e.g., Shalon et al., supra; and Chee et al., supra).

Optimal hybridization conditions will depend on the length (e.g., oligomer versus polynucleotide greater than 200 bases) and type (e.g., RNA, DNA, PNA) of labeled probe and immobilized polynticleotide or oligonucleotide. When cDNA microarrays, such as those described by Schena et al. are used, typical hybridization conditions are hybridization in 5×SSC plus 0.2% SDS at 65° C. for 4 hours, followed by washes at 25° C. in low stringency wash buffer (e.g., 1×SSC plus 0.2% SDS) followed by 10 minutes at 25° C. in high stringency wash buffer (0.1×SSC plus 0.2% SDS). See, Shena et al., Proc. Natl. Acad. Sci. USA 1996, 93:10614). Useful hybridization conditions are also provided in, e.g., Tijessen, 1993, Hybridization With Nucleic Acid Probes, Elsevier Science Publishers B.V. See, also, Kricka, 1992, Nonisotopic DNA Probe Techniques, Academic Press, San Diego, Calif.

Signal Detection and Analysis. When fluorescently labeled probes are used, the fluorescence emissions at each site of a transcript microarray can be preferably detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see, Shalon et al., Genome Research 1996, 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes.

Fluorescence laser scanning devices are described in Schena et al., Genome Res. 1996, 6:639-645 and in other references cited herein Alternatively, the fiber-optic bundle described by Ferguson et al., Nature Biotech. 1996, 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated.

In one preferred embodiment of the invention, the relative abundance of an mRNA in two cells or cell lines tested (e.g., in a treated verses untreated cell) may be scored as perturbed (i.e., where the abundance is different in the two sources of mRNA tested) or as not perturbed (i.e., where the relative abundance in the two sources is the same or is unchanged). Preferably, the difference is scored as perturbed if the difference between the two sources of RNA of at least a factor of about 25% (i.e., RNA from one sources is about 25% more abundant than in the other source), more preferably about 50%. Still more preferably, the RNA may be scored as perturbed when the difference between the two sources of RNA is at least about a factor of two. Indeed, the difference in abundance between the two sources may be by a factor of three, of five, or more.

In other embodiments, it may be advantageous also to determine the magnitude of the perturbation. This may be done, as noted above, by calculating the ratio of the emission of the two fluorophores used for differential labeling, or by analogous methods that will be readily apparent to those of skill in the art.

Experiments which are the basis of the invention are described in the following example, with reference to the accompanying drawings in which:

FIG. 1 shows mitochondrial changes associated with schizophrenia;

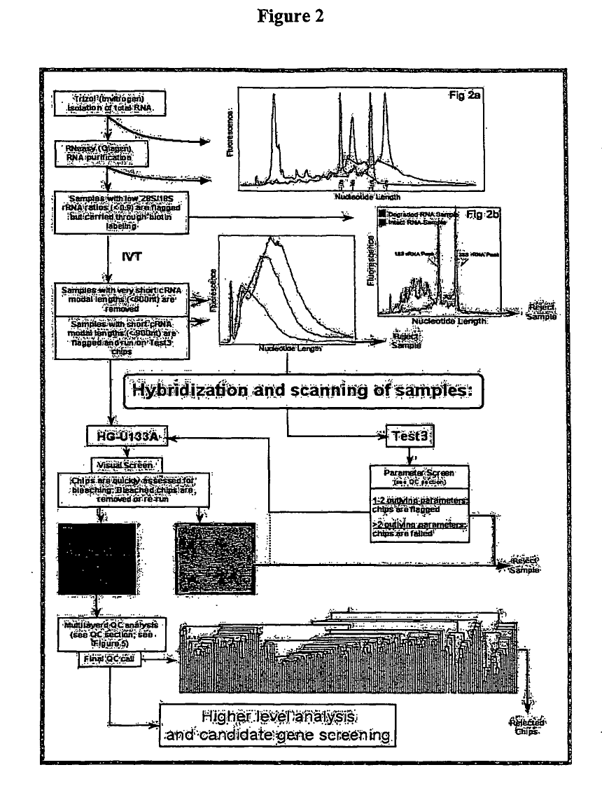

FIG. 2 shows sample quality control steps;

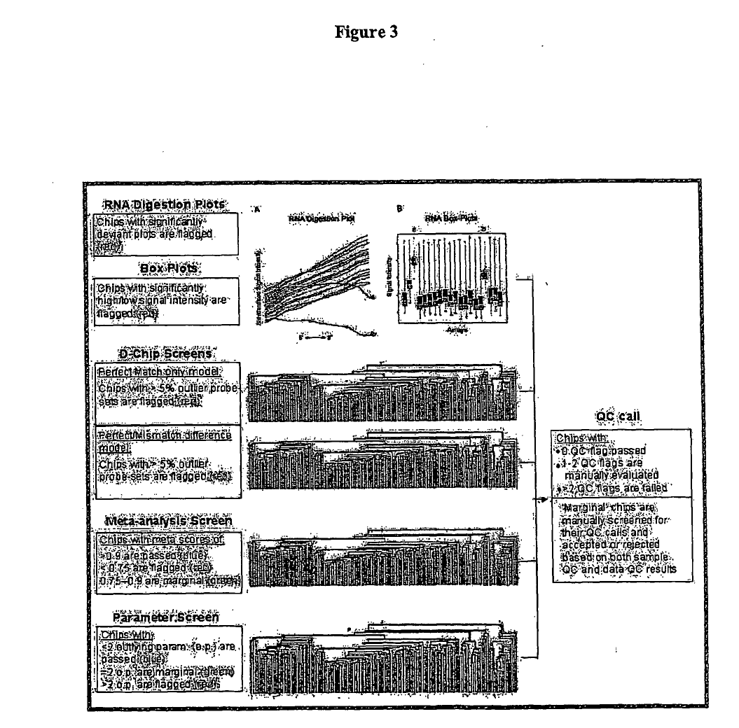

FIG. 3 shows data quality control steps;

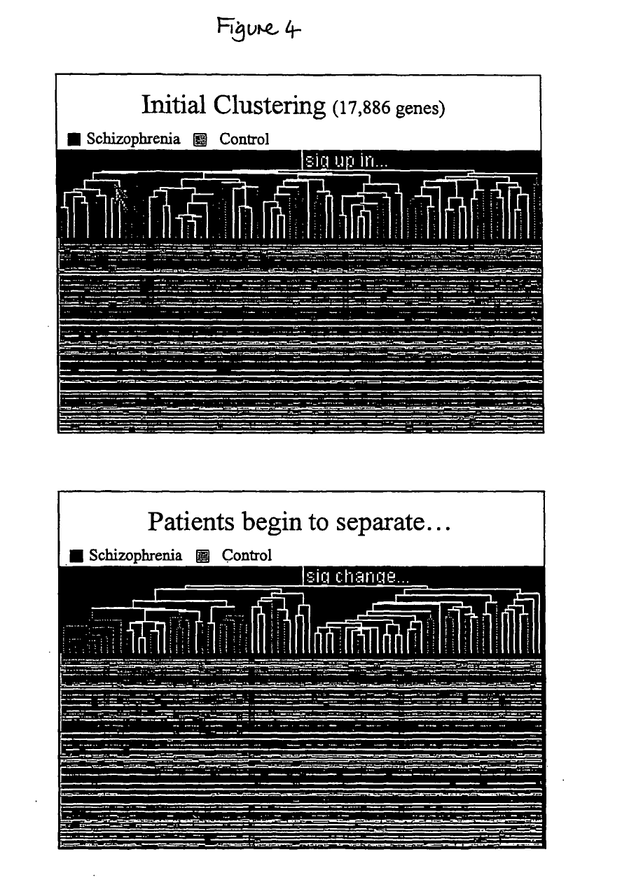

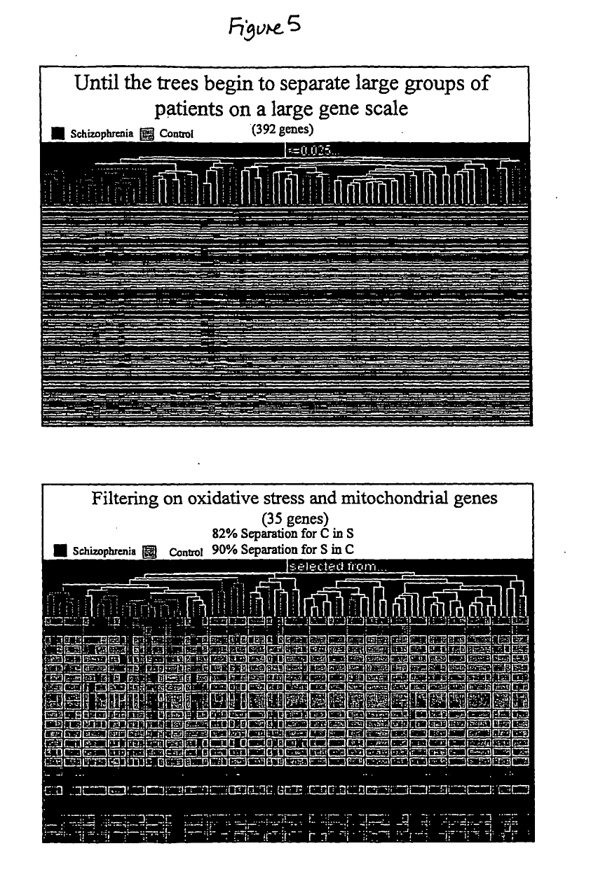

FIGS. 4 and 5 show clustering analysis between control (C) and schizophrenia (S) samples; and

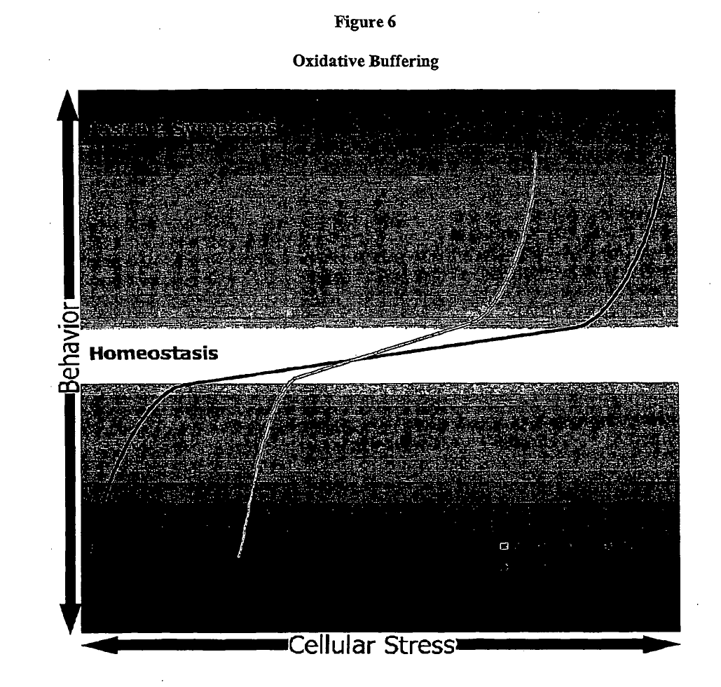

FIG. 6 shows oxidative buffering.

### EXAMPLE

Integrating Transcriptomics, Proteomics, and Classical Genetics:

Fishing in Modern Neuropsychiatric Research

Affymetrix® GeneChip® Post-Mortem Brain Studies

| HG-U133 set includes: | Our Studies: |
|---|---|
| 39,000 probes | 150 PM human brain samples from |
| 33,000 annotated | SMRI |
| 2 chips: A and B | Completed on HG-U133A chips |
| Each w/ ˜23,000 genes on 1.28 cm² | and continuing on B |
| | Extensive Quality Control (QC) |
| | steps |
| | Cluster analysis |

Sample QC Steps (see FIG. 2):
Total RNA is screened for degraded samples
cRNA is generated and screened for poor modal length
Poor samples are run on Test3 GeneChips®
Prisitine samples are run on U133 GeneChips®
   Microarrays are put through our in-house Data QC screen and only “clean” data sets are retained, poor set samples are rerun or rejected
   Data QC Steps (see FIG. 3):

6 data filters
RNA digestion plots
Box plots
2 D-chip screens
In-house parameter script
In-house heuristic meta-analysis script
   Data Mining

Flag Filtering

Fold Difference and Significance Filtering

Subset Significant Gene Overlapping

Pathway Specific Filtering

Cluster Analysis (see FIGS. 4 and 5)

Initial Clustering (17,886 genes)

Patients begin to separate . . .

Until the trees begin to separate large groups of patients on a large gene scale (392 genes)

Filtering on oxidative stress and mitochondrial genes (35 genes)

82% separation for C in S
90% separation for S in C
   Mitochondrial Involvement: Evidence for ROS Stress (see FIG. 6)
   Oxidative Stress:
   Evidence for Stress Response
   Up-regulations in MT transcripts

Changes in specific ROS stress systems including:

| | |
|---|---|
| SOD's | HIF's |
| MSR | Fe containing molecules |
| GLRX | |
| PDCD's | |
| Specific RAS pathways | |

Changes in DNA repair mechanisms
Future Directions

Continue data mining of Affymetrix® results

Validate gene hits via Q-PCR and poly-“omics”

Genotyping and SNP analysis of genes that separate patient groups

GeneChip analysis of peripheral tissues including liver, spleen, blood and duramata

TABLE 1

| | | | | | | GeneSpring | | | Dchip | | | |
|---|---|---|---|---|---|---|---|---|---|---|---|---|
| | | | | | | Norm | | | Norm | | | |
| | | | | | | Fold | | ttest | Fold | | ttest | |
| | Systematic | Common | Genbank | Map | UniGene | S | | S-C | S | | S-C | Description |
| Significant | 205060_at | PARG | NM_003631 | 10q11.23 | Hs.91390 | 1.510748 | Down | 3.7332E−05 | 1.1861727 | Down | 0.0160033 | Homo sapiens poly (ADP- |
| clustering 90% | | | | | | | | | | | | ribose) glycohydrolase (PARG), |
| separation of | | | | | | | | | | | | mRNA. |
| schizophrenics | 211662_s_at | VDAC2 | L08666 | 10q22 | Hs.78902 | 1.12617 | Down | 0.014133997 | 1.1240973 | Down | 0.0034869 | Homo sapiens voltage- |
| from controls | | | | | | | | | | | | dependent anion channel 2 |
| | | | | | | | | | | | | (VDAC2), mRNA. |
| | 210004_at | OLR1; OLR1; | AF035776 | 12p13.2-p12.3 | Hs.77729 | 1.377409 | Down | 0.004075548 | 1.2097276 | Down | 0.0027743 | synonyms: LOX1, LOX-1, |
| | | LOX1; LOX-1; | | | | | | | | | | SCARE1; scavenger receptor |
| | | SCARE1 | | | | | | | | | | class E, member 1; Homo |
| | | | | | | | | | | | | sapiens oxidised low density |
| | | | | | | | | | | | | lipoprotein (lectin-like) receptor |
| | | | | | | | | | | | | 1 (OLR1), mRNA. |
| | 208736_at | ARPC3; | AF004561 | 12q24.11 | Hs.293750 | 1.285561 | Down | 5.21013E−05 | 1.1353506 | Down | 0.0402546 | synonyms: ARC21, p21-Arc; |
| | | ARPC3; | | | | | | | | | | ARP2/3 protein complex subunit |
| | | ARC21; p21-Arc | | | | | | | | | | p21; Homo sapiens actin related |
| | | | | | | | | | | | | protein 2/3 complex, subunit 3, |
| | | | | | | | | | | | | 21 kDa (ARPC3), mRNA. |
| | 208909_at | UQCRFS1; | BC000649 | 19q12-q13.1 | Hs.3712 | 1.168573 | Down | 0.01259279 | 1.2110709 | Down | 0.0002244 | synonym: RIS1; Homo sapiens |
| | | UQCRFS1; | | | | | | | | | | ubiquinol-cytochrome c |
| | | RIS1 | | | | | | | | | | reductase, Rieske iron-sulfur |
| | | | | | | | | | | | | polypeptide 1 (UQCRFS1), |
| | | | | | | | | | | | | nuclear gene encoding |
| | | | | | | | | | | | | mitochondrial protein, mRNA. |
| | 217976_s_at | DNCLI1 | NM_016141 | | Hs.266483 | 1.334079 | Down | 6.56512E−05 | 1.2670291 | Down | 0.0003879 | Homo sapiens dynein, |
| | | | | | | | | | | | | cytoplasmic, light intermediate |
| | | | | | | | | | | | | polypeptide 1 (DNCLI1), mRNA. |
| | 203966_s_at | PPM1A; | NM_021003 | | Hs.57764 | 1.331601 | Down | 3.751E−05 | 1.1790799 | Down | 0.0026358 | synonyms: PP2CA, PP2C- |
| | | PPM1A; | | | | | | | | | | ALPHA, MGC9201; isoform 1 is |
| | | PP2CA; | | | | | | | | | | encoded by transcript variant 1 |
| | | MGC9201; | | | | | | | | | | and 3; protein phosphatase 2C |
| | | PP2C-ALPHA | | | | | | | | | | alpha isoform; Homo sapiens |
| | | | | | | | | | | | | protein phosphatase 1A |
| | | | | | | | | | | | | (formerly 2C), magnesium- |
| | | | | | | | | | | | | dependent, alpha isoform |
| | | | | | | | | | | | | (PPM1A), transcript variant 1, |
| | | | | | | | | | | | | mRNA.; synonyms: PP2CA, |
| | | | | | | | | | | | | PP2C-ALPHA, MGC9201; |
| | | | | | | | | | | | | isoform 2 is encoded by |
| | | | | | | | | | | | | transcript variant 2; protein |
| | | | | | | | | | | | | phosphatase 2C alpha isoform; |
| | | | | | | | | | | | | Homo sapiens protein |
| | | | | | | | | | | | | phosphatase 1A (formerly 2C), |
| | | | | | | | | | | | | magnesium-dependent, alpha |
| | | | | | | | | | | | | isoform (PPM1A), transcript |
| | | | | | | | | | | | | variant 2, mRNA.; synonyms: |
| | | | | | | | | | | | | PP2CA, PP2C-ALPHA, |
| | | | | | | | | | | | | MGC9201; isoform 1 is encoded |
| | | | | | | | | | | | | by transcript variant 1 and 3; |
| | | | | | | | | | | | | protein phosphatase 2C alpha |
| | | | | | | | | | | | | isoform; Homo sapiens protein |
| | | | | | | | | | | | | phosphatase 1A (formerly 2C), |
| | | | | | | | | | | | | magnesium-dependent, alpha |
| | | | | | | | | | | | | isoform (PPM1A), transcript |
| | 218671_s_at | ATPIF1; | NM_016311 | 1p35.3 | Hs.241336 | 1.158111 | Down | 0.016292648 | 1.1556271 | Down | 0.0163928 | synonyms: MGC8898, |
| | | ATPIF1; | | | | | | | | | | MGC1167, ATPI; isoform 1 is |
| | | MGC1167; | | | | | | | | | | encoded by transcript variant 1; |
| | | MGC8898 | | | | | | | | | | Homo sapiens ATPase inhibitory |
| | | | | | | | | | | | | factor 1 (ATPIF1), transcript |
| | | | | | | | | | | | | variant 1, nuclear gene encoding |
| | | | | | | | | | | | | mitochondrial protein, mRNA.; |
| | | | | | | | | | | | | synonyms: MGC8898, |
| | | | | | | | | | | | | MGC1167, ATPI; isoform 2 is |
| | | | | | | | | | | | | encoded by transcript variant 2; |
| | | | | | | | | | | | | Homo sapiens ATPase inhibitory |
| | | | | | | | | | | | | factor 1 (ATPIF1), transcript |
| | | | | | | | | | | | | variant 2, nuclear gene encoding |
| | | | | | | | | | | | | mitochondrial protein, mRNA.; |
| | | | | | | | | | | | | synonyms: MGC8898, |
| | | | | | | | | | | | | MGC1167, ATPI; isoform 3 is |
| | | | | | | | | | | | | encoded by transcript variant 3; |
| | | | | | | | | | | | | Homo sapiens ATPase inhibitory |
| | | | | | | | | | | | | factor 1 (ATPIF1), transcript |
| | | | | | | | | | | | | variant 3, nuclear gene encoding |
| | | | | | | | | | | | | mitochondrial protein, mRNA. |
| | 219933_at | GLRX2; GLRX2; | NM_016066 | 1q31.2-q31.3 | Hs.5054 | 1.214851 | Down | 0.000850749 | 1.237224 | Down | 0.0001692 | synonym: GRX2; |
| | | GRX2 | | | | | | | | | | thioltransferase; contains |
| | | | | | | | | | | | | nuclear membrane localisation; |
| | | | | | | | | | | | | CGI-133 protein; Homo sapiens |
| | | | | | | | | | | | | glutaredoxin 2 (GLRX2), mRNA. |
| | 215171_s_at | TIMM17A; | AK023063 | 1q32.1 | Hs.20716 | 1.223866 | Down | 0.000427331 | 1.2024004 | Down | 0.0004325 | synonyms: TIM17, TIM17A; |
| | | TIMM17A; | | | | | | | | | | preprotein translocase; Homo |
| | | TIM17; TIM17A | | | | | | | | | | sapiens translocase of inner |
| | | | | | | | | | | | | mitochondrial membrane 17 |
| | | | | | | | | | | | | homolog A (yeast) (TIMM17A), |
| | | | | | | | | | | | | mRNA. |
| | 210418_s_at | IDH3B; IDH3B; | AF023265 | 20p13 | Hs.155410 | 1.157609 | Down | 0.009473711 | 1.1832137 | Down | 0.0036835 | synonyms: H-IDHB, MGC903, |
| | | H-IDHB; | | | | | | | | | | FLJ11043; isocitric |
| | | MGC903; | | | | | | | | | | dehydrogenase; NAD+-specific |
| | | FLJ11043 | | | | | | | | | | isocitrate dehydrogenase beta |
| | | | | | | | | | | | | precursor; NAD+-specific |
| | | | | | | | | | | | | isocitrate dehydrogenase b |
| | | | | | | | | | | | | subunit; NAD+-specific ICDH; |
| | | | | | | | | | | | | isocitrate dehydrogenase, |
| | | | | | | | | | | | | NAD(+)-specific, mitochondrial, |
| | | | | | | | | | | | | beta subunit; Homo sapiens |
| | | | | | | | | | | | | isocitrate dehydrogenase 3 |
| | | | | | | | | | | | | (NAD+) beta (IDH3B), nuclear |
| | | | | | | | | | | | | gene encoding mitochondrial |
| | | | | | | | | | | | | protein, transcript variant 1, |
| | | | | | | | | | | | | mRNA. |
| | 203946_s_at | ARG2 | U75667 | 14q24.1-q24.3 | Hs.172851 | 1.241001 | Down | 0.006993609 | 1.1794939 | Down | 0.0060619 | kidney arginase; nonhepatic |
| | | | | | | | | | | | | arginase; L-arginine |
| | | | | | | | | | | | | amidinohydrolase; L-arginine |
| | | | | | | | | | | | | ureahydrolase; A-II; Homo |
| | | | | | | | | | | | | sapiens arginase, type II |
| | | | | | | | | | | | | (ARG2), nuclear gene encoding |
| | | | | | | | | | | | | mitochondrial protein, mRNA. |
| | 200880_at | DNAJA1 | AL534104 | 9p13-p12 | Hs.94 | 1.255564 | Down | 0.000175313 | 1.2076009 | Down | 5.252E−05 | DnaJ (Hsp40) homolog, |
| | | | | | | | | | | | | subfamily A, member 1 |
| | 213921_at | SST; SST; | NM_001048 | 3q28 | Hs.12409 | 1.515051 | Down | 0.000215427 | 1.536124 | Down | 7.312E−05 | synonym: SMST; Homo sapiens |
| | | SMST | | | | | | | | | | somatostatin (SST), mRNA. |
| | 210014_x_at | IDH3B; IDH3B; | AF023266 | 20p13 | Hs.155410 | 1.122301 | Down | 0.019203203 | 1.121091 | Down | 0.01916 | synonyms: H-IDHB, MGC903, |
| | | H-IDHB; | | | | | | | | | | FLJ11043; isocitric |
| | | MGC903; | | | | | | | | | | dehydrogenase; NAD+-specific |
| | | FLJ11043 | | | | | | | | | | isocitrate dehydrogenase beta |
| | | | | | | | | | | | | precursor; NAD+-specific |
| | | | | | | | | | | | | isocitrate dehydrogenase b |
| | | | | | | | | | | | | subunit; NAD+-specific ICDH; |
| | | | | | | | | | | | | isocitrate dehydrogenase, |
| | | | | | | | | | | | | NAD(+)-specific, mitochondrial, |
| | | | | | | | | | | | | beta subunit; Homo sapiens |
| | | | | | | | | | | | | isocitrate dehydrogenase 3 |
| | | | | | | | | | | | | (NAD+) beta (IDH3B), nuclear |
| | | | | | | | | | | | | gene encoding mitochondrial |
| | | | | | | | | | | | | protein, transcript variant 1, |
| | | | | | | | | | | | | mRNA. |
| | 220045_at | NEUROD6; | NM_022728 | | Hs.45152 | 1.267805 | Down | 0.008816781 | 1.3729296 | Down | 8.878E−05 | synonyms: Atoh2, NEX1M, Math |
| | | NEUROD6; | | | | | | | | | | 2; Homo sapiens neurogenic |
| | | Atoh2; NEX1M; | | | | | | | | | | differentiation 6 (NEUROD6), |
| | | Math-2 | | | | | | | | | | mRNA. |
| | 203336_s_at | ICAP-1A | AL548363 | 2p25.2 | Hs.173274 | 1.316151 | Down | 0.000466967 | 1.3571769 | Down | 1.818E−05 | integrin cytoplasmic domain- |
| | | | | | | | | | | | | associated protein 1 |
| | 218289_s_at | FLJ23251 | NM_024818 | 3q22.1 | Hs.170737 | 1.15484 | Down | 0.022580668 | 1.1592489 | Down | 0.0060202 | Homo sapiens hypothetical |
| | | | | | | | | | | | | protein FLJ23251 (FLJ23251), |
| | | | | | | | | | | | | mRNA. |
| | 204679_at | KCNK1; | NM_002245 | 1q42-q43 | Hs.79351 | 1.253524 | Down | 0.000834664 | 1.2688284 | Down | 3.552E−05 | synonyms: DPK, HOHO, |
| | | KCNK1; DPK; | | | | | | | | | | TWIK1, TWIK-1; potassium |
| | | HOHO; TWIK1; | | | | | | | | | | inwardly-rectifying channel, |
| | | TWIK-1 | | | | | | | | | | subfamily K, member 1; |
| | | | | | | | | | | | | potassium channel, subfamily K, |
| | | | | | | | | | | | | member 1 (TWIK-1); Homo |
| | | | | | | | | | | | | sapiens potassium channel, |
| | | | | | | | | | | | | subfamily K, member 1 |
| | | | | | | | | | | | | (KCNK1), mRNA. |
| | 202825_at | SLC25A4; | NM_001151 | 4q35 | Hs.2043 | 1.243055 | Down | 0.003755193 | 1.1702685 | Down | 0.004136 | synonyms: T1, ANT, ANT1, |
| | | SLC25A4; T1; | | | | | | | | | | PEO2, PEO3; adenine |
| | | ANT; ANT1; | | | | | | | | | | nucleotide translocator 1 |
| | | PEO2; PEO3 | | | | | | | | | | (skeletal muscle); Homo sapiens |
| | | | | | | | | | | | | solute carrier family 25 |
| | | | | | | | | | | | | (mitochondrial carrier; adenine |
| | | | | | | | | | | | | nucleotide translocator), |
| | | | | | | | | | | | | member 4 (SLC25A4), nuclear |
| | | | | | | | | | | | | gene encoding mitochondrial |
| | | | | | | | | | | | | protein, mRNA. |
| | 218674_at | FLJ13611 | NM_024941 | 5q12.2 | Hs.282958 | 1.280891 | Down | 3.93672E−05 | 1.1867894 | Down | 0.0110943 | Homo sapiens hypothetical |
| | | | | | | | | | | | | protein FLJ13611 (FLJ13611), |
| | | | | | | | | | | | | mRNA. |
| | 218946_at | HIRIP5; HIRIP5; | NM_015700 | 2p15-p13 | Hs.430439 | 1.20213 | Down | 0.000960488 | 1.2521306 | Down | 9.482E−05 | synonym: CGI-33; HIRIP5 |
| | | CGI-33 | | | | | | | | | | protein; HIRA-interacting protein |
| | | | | | | | | | | | | 5; Homo sapiens HIRA |
| | | | | | | | | | | | | interacting protein 5 (HIRIP5), |
| | | | | | | | | | | | | mRNA. |
| | 201597_at | COX7A2; | NM_001865 | 6q12 | Hs.70312 | 1.149356 | Down | 0.00796477 | 1.182428 | Down | 1.847E−05 | synonyms: COX7AL, COX7AL1, |
| | | COX7A2; | | | | | | | | | | COXVIIa-L; hepatic cytochrome- |
| | | COX7AL; | | | | | | | | | | c oxidase chain VIIa; Homo |
| | | COX7AL1; | | | | | | | | | | sapiens cytochrome c oxidase |
| | | COXVIIa-L | | | | | | | | | | subunit VIIa polypeptide 2 (liver) |
| | | | | | | | | | | | | (COX7A2), nuclear gene |
| | | | | | | | | | | | | encoding mitochondrial protein, |
| | | | | | | | | | | | | mRNA. |
| | 203663_s_at | COX5A; | NM_004255 | 15q25 | Hs.323834 | 1.197943 | Down | 0.004379827 | 1.2294325 | Down | 7.246E−05 | synonyms: VA, COX, COX-VA; |
| | | COX5A; VA; | | | | | | | | | | cytochrome c oxidase |
| | | COX-VA | | | | | | | | | | polypeptide, mitochondrial |
| | | | | | | | | | | | | precursor; Homo sapiens |
| | | | | | | | | | | | | cytochrome c oxidase subunit |
| | | | | | | | | | | | | Va (COX5A), nuclear gene |
| | | | | | | | | | | | | encoding mitochondrial protein, |
| | | | | | | | | | | | | mRNA. |
| | 206552_s_at | TAC1; TAC1; | NM_003182 | 7q21-q22 | Hs.2563 | 1.662809 | Down | 2.27281E−07 | 1.5689365 | Down | 2.145E−06 | synonyms: NK2, NKNA, TAC2; |
| | | NK2; NKNA; | | | | | | | | | | neurokinin A; neurokinin alpha; |
| | | TAC2 | | | | | | | | | | tachykinin 2; substance K; |
| | | | | | | | | | | | | neuropeptide K; neuropeptide |
| | | | | | | | | | | | | gamma; substance P; |
| | | | | | | | | | | | | neurokinin 1; neurokinin 2; |
| | | | | | | | | | | | | neuromedin L; Homo sapiens |
| | | | | | | | | | | | | tachykinin, precursor 1 |
| | | | | | | | | | | | | (substance K, substance P, |
| | | | | | | | | | | | | neurokinin 1, neurokinin 2, |
| | | | | | | | | | | | | neuromedin L, neurokinin alpha, |
| | | | | | | | | | | | | neuropeptide K, neuropeptide |
| | | | | | | | | | | | | gamma) (TAC1), transcript |
| | | | | | | | | | | | | variant beta, mRNA.; synonyms: |
| | | | | | | | | | | | | NK2, NKNA, TAC2; neurokinin |
| | | | | | | | | | | | | A; neurokinin alpha; tachykinin |
| | | | | | | | | | | | | 2; substance K; neuropeptide K; |
| | | | | | | | | | | | | neuropeptide gamma; |
| | | | | | | | | | | | | substance P; neurokinin 1; |
| | | | | | | | | | | | | neurokinin 2; neuromedin L; |
| | | | | | | | | | | | | Homo sapiens tachykinin, |
| | | | | | | | | | | | | precursor 1 (substance K, |
| | | | | | | | | | | | | substance P, neurokinin 1, |
| | | | | | | | | | | | | neurokinin 2, neuromedin L, |
| | | | | | | | | | | | | neurokinin alpha, neuropeptide |
| | | | | | | | | | | | | K, neuropeptide gamma) |
| | | | | | | | | | | | | (TAC1), transcript variant alpha, |
| | 202233_s_at | UQCRH | NM_006004 | | Hs.73818 | 1.148508 | Down | 0.025106627 | 1.1682167 | Down | 3.687E−05 | Homo sapiens ubiquinol- |
| | | | | | | | | | | | | cytochrome c reductase hinge |
| | | | | | | | | | | | | protein (UQCRH), mRNA. |
| | 218573_at | MAGEH1; | NM_014061 | Xp11.22 | Hs.279819 | 1.167834 | Down | 0.018662092 | 1.2202681 | Down | 9.034E−05 | synonym: APR-1; restin; MAGEH1 |
| | | MAGEH1; APR-1 | | | | | | | | | | antigen; Homo sapiens APR- |
| | | | | | | | | | | | | 1 protein (MAGEH1), mRNA. |
| | 217769_s_at | C13orf12; | NM_015932 | 13q12.13 | Hs.279813 | 1.144417 | Down | 0.009888431 | 1.1710335 | Down | 5.867E−05 | synonyms: HSPC014, |
| | | C13orf12; | | | | | | | | | | 2510048O06Rik; Homo sapiens |
| | | HSPC014; | | | | | | | | | | chromosome 13 open reading |
| | | 2510048O06Rik | | | | | | | | | | frame 12 (C13orf12), mRNA. |
| | 201323_at | EBNA1BP2; | NM_006824 | 1p35-p33 | Hs.346868 | 1.13658 | Down | 0.018139154 | 1.1689036 | Down | 4.837E−05 | synonyms: P40, EBP2, NOBP; |
| | | EBNA1BP2; | | | | | | | | | | cell proliferation-associated |
| | | P40; EBP2; | | | | | | | | | | protein; nucleolar protein p40; |
| | | NOBP | | | | | | | | | | homolog of yeast EBNA1- |
| | | | | | | | | | | | | binding protein; nuclear FGF3 |
| | | | | | | | | | | | | binding protein; EBNA1-binding |
| | | | | | | | | | | | | protein 2; Homo sapiens EBNA1 |
| | | | | | | | | | | | | binding protein 2 (EBNA1BP2), |
| | | | | | | | | | | | | mRNA. |
| | 219619_at | DIRAS2; | NM_017594 | 9q22.1 | Hs.165636 | 1.28122 | Down | 3.39475E−05 | 1.2324031 | Down | 1.828E−05 | synonyms: Di-Ras2, |
| | | DIRAS2; Di- | | | | | | | | | | DKFZp761C07121; member of |
| | | Ras2; | | | | | | | | | | the Ras familysmall GTP- |
| | | DKFZp761C07121 | | | | | | | | | | binding protein; Homo sapiens |
| | | | | | | | | | | | | DIRAS family, GTP-binding RAS |
| | | | | | | | | | | | | like 2 (DIRAS2), mRNA. |
| | 213924_at | MPPE1 | BF476502 | 18p11.21 | Hs.154145 | 1.19464 | Down | 0.004053367 | 1.1134946 | Down | 0.0862616 | metallo phosphoesterase |
| | 218255_s_at | FBS1; FBS1; | NM_022452 | 16p11.2 | Hs.77735 | 1.308398 | Up | 5.96696E−05 | 1.2807998 | Up | 4.124E−05 | synonyms: FBS, FLJ11618; |
| | | FLJ11618 | | | | | | | | | | likely ortholog of mouse fibrosin; |
| | | | | | | | | | | | | Homo sapiens fibrosin 1 (FBS1), |
| | | | | | | | | | | | | mRNA. |
| | 202908_at | WFS1; WFS1; | NM_006005 | 4p16 | Hs.26077 | 1.264465 | Up | 1.9637E−05 | 1.2121222 | Up | 0.0018794 | synonyms: WFS, WFRS, |
| | | WFRS; DFNA6; | | | | | | | | | | DFNA6, DFNA14, DFNA38, |
| | | DFNA14; | | | | | | | | | | DIDMOAD, WOLFRAMIN; |
| | | DFNA38; | | | | | | | | | | Homo sapiens Wolfram |
| | | DIDMOAD; | | | | | | | | | | syndrome 1 (wolframin) |
| | | WOLFRAMIN | | | | | | | | | | (WFS1), mRNA. |
| | 214203_s_at | PRODH | AA074145 | 22q11.21 | Hs.343874 | 1.410089 | Up | 0.003560936 | 1.1914315 | Up | 0.0336675 | proline dehydrogenase |
| | | | | | | | | | | | | (oxidase) 1 |
| | 204294_at | AMT; AMT; | NM_000481 | 3p21.2-p21.1 | Hs.102 | 1.20989 | Up | 0.003991536 | 1.0981119 | Up | 0.0102842 | synonyms: GCE, NKH, GCST; |
| | | GCE; NKH; | | | | | | | | | | Homo sapiens |
| | | GCST | | | | | | | | | | aminomethyltransferase (glycine |
| | | | | | | | | | | | | cleavage system protein T) |
| | | | | | | | | | | | | (AMT), mRNA. |
| | 209275_s_at | CLN3; CLN3; | AF015593 | 16p12.1 | Hs.194660 | 1.194657 | Up | 0.010213986 | 1.1260952 | Up | 0.0775445 | synonym: BTS; Homo sapiens |
| | | BTS | | | | | | | | | | ceroid-lipofuscinosis, neuronal |
| | | | | | | | | | | | | 3, juvenile (Batten, Spielmeyer- |
| | | | | | | | | | | | | Vogt disease) (CLN3), mRNA. |
| | 209600_s_at | ACOX1; | S69189 | 17q24-17q25 | Hs.379991 | 1.271366 | Up | 0.003365436 | 1.1690975 | Up | 0.0599128 | synonyms: ACOX, PALMCOX, |
| | | ACOX1; | | | | | | | | | | MGC1198; acyl-coenzyme A |
| | | MGC1198; | | | | | | | | | | oxidase 1; Homo sapiens acyl- |
| | | PALMCOX | | | | | | | | | | Coenzyme A oxidase 1, |
| | | | | | | | | | | | | palmitoyl (ACOX1), transcript |
| | | | | | | | | | | | | variant 1, mRNA.; synonyms: |
| | | | | | | | | | | | | ACOX, PALMCOX, MGC1198; |
| | | | | | | | | | | | | acyl-coenzyme A oxidase 1; |
| | | | | | | | | | | | | Homo sapiens acyl-Coenzyme A |
| | | | | | | | | | | | | oxidase 1, palmitoyl (ACOX1), |
| | | | | | | | | | | | | transcript variant 2, mRNA. |
| | 202275_at | G6PD; G6PD; | NM_000402 | Xq28 | Hs.80206 | 1.327344 | Up | 0.009268356 | 1.1621796 | Up | 0.0821313 | synonym: G6PD1; Homo |
| | | G6PD1 | | | | | | | | | | sapiens glucose-6-phosphate |
| | | | | | | | | | | | | dehydrogenase (G6PD), nuclear |
| | | | | | | | | | | | | gene encoding mitochondrial |
| | | | | | | | | | | | | protein, mRNA. |
| | 208369_s_at | GCDH | NM_013976 | 19p13.2 | Hs.184141 | 1.179371 | Up | 0.004779667 | 1.0740626 | Up | 0.0802165 | Homo sapiens glutaryl- |
| | | | | | | | | | | | | Coenzyme A dehydrogenase |
| | | | | | | | | | | | | (GCDH), nuclear gene encoding |
| | | | | | | | | | | | | mitochondrial protein, transcript |
| | | | | | | | | | | | | variant 1, mRNA.; Homo |
| | | | | | | | | | | | | sapiens glutaryl-Coenzyme A |
| | | | | | | | | | | | | dehydrogenase (GCDH), |
| | | | | | | | | | | | | nuclear gene encoding |
| | | | | | | | | | | | | mitochondrial protein, transcript |
| | | | | | | | | | | | | variant 2, mRNA. |
| | 213818_x_at | COL5A1 | AI862325 | 11 | Hs.381134 | 1.562872 | Up | 3.03281E−05 | 1.1283786 | Up | 0.20423431 | ESTs, Moderately similar to |
| | | | | | | | | | | | | RIKEN cDNA 1810059G22 [Mus |
| | | | | | | | | | | | | musculus] [M. musculus] |
| | 214892_x_at | NY-REN-24 | BC004262 | 19p13.3 | Hs.128425 | 1.290386 | Up | 3.8958E−05 | 1.0396863 | Up | 0.43972711 | NY-REN-24 antigen |
| | 215568_x_at | HMGCL; | AL031295 | 1p36.1-p35 | Hs.831 | 1.283746 | Up | 3.92631E−05 | 1.0334069 | Up | 0.5203439 | synonym: HL; 3-hydroxy-3- |
| | | HMGCL; HL | | | | | | | | | | methylglutaryl-Coenzyme A |
| | | | | | | | | | | | | lyase; 3-hydroxy-3- |
| | | | | | | | | | | | | methylglutaryl-Coenzyme A |
| | | | | | | | | | | | | lyase |
| | | | | | | | | | | | | (hydroxymethylglutaricaciduria); |
| | | | | | | | | | | | | Homo sapiens 3-hydroxymethyl- |
| | | | | | | | | | | | | 3-methylglutaryl-Coenzyme A |
| | | | | | | | | | | | | lyase |
| | | | | | | | | | | | | (hydroxymethylglutaricaciduria) |
| | | | | | | | | | | | | (HMGCL), mRNA. |
| | 207506_at | TXNL2; TXNL2; | NM_006541 | 6p25.3 | Hs.42644 | 1.375835 | Up | 0.005318749 | 1.0210061 | Up | 0.66528908 | synonym: PICOT; PKC- |
| | | PICOT | | | | | | | | | | interacting cousin of thioredoxin; |
| | | | | | | | | | | | | Homo sapiens thioredoxin-like 2 |
| | | | | | | | | | | | | (TXNL2), mRNA. |
| | 205236_x_at | SOD3 | NM_003102 | 4p16.3-q21 | Hs.2420 | 1.290517 | Up | 0.001685857 | 1.0086441 | Up | 0.88220476 | Homo sapiens superoxide |
| | | | | | | | | | | | | dismutase 3, extracellular |
| | | | | | | | | | | | | (SOD3), mRNA. |
| | 203576_at | BCAT2; BCAT2; | NM_001190 | 19q13 | Hs.101408 | 1.287101 | Up | 0.000421341 | 1.0767065 | Up | 0.19972382 | synonym: BCT2; predicted |
| | | BCT2 | | | | | | | | | | mature protein begins at amino |
| | | | | | | | | | | | | acid 28; Homo sapiens |
| | | | | | | | | | | | | branched chain |
| | | | | | | | | | | | | aminotransferase 2, |
| | | | | | | | | | | | | mitochondrial (BCAT2), mRNA. |
| | 208581_x_at | MT1X | NM_005952 | 16q13 | Hs.374950 | 1.547521 | Up | 0.010241902 | 1.3210147 | Up | 0.0043797 | synonyms: MT1, MT-1I; Homo |
| | | | | | | | | | | | | sapiens metallothionein 1X |
| | | | | | | | | | | | | (MT1X), mRNA. |
| purine | 203722_at | ALDH4A1; | NM_003748 | 1p36 | Hs.77448 | 1.432904 | Up | 0.021909934 | 1.2649056 | Up | 0.07248872 | synonyms: P5CD, ALDH4, |
| metabolism | | ALDH4A1; | | | | | | | | | | P5CDH, P5CDhL, P5CDhS; |
| (matrix) | | P5CD; P5CDH; | | | | | | | | | | aldehyde dehydrogenase 4; |
| | | P5CDh; | | | | | | | | | | mitochondrial delta-1-pyrroline 5- |
| | | P5CDhL; | | | | | | | | | | carboxylate dehydrogenase; |
| | | P5CDhS | | | | | | | | | | P5C dehydrogenase; Homo |
| | | | | | | | | | | | | sapiens aldehyde |
| | | | | | | | | | | | | dehydrogenase 4 family, |
| | | | | | | | | | | | | member A1 (ALDH4A1), nuclear |
| | | | | | | | | | | | | gene encoding mitochondrial |
| | | | | | | | | | | | | protein, transcript variant |
| | | | | | | | | | | | | P5CDhL, mRNA.; synonyms: |
| | | | | | | | | | | | | P5CD, ALDH4, P5CDH, |
| | | | | | | | | | | | | P5CDhL, P5CDhS; aldehyde |
| | | | | | | | | | | | | dehydrogenase 4; mitochondrial |
| | | | | | | | | | | | | delta-1-pyrroline 5-carboxylate |
| | | | | | | | | | | | | dehydrogenase; P5C |
| | | | | | | | | | | | | dehydrogenase; Homo sapiens |
| | | | | | | | | | | | | aldehyde dehydrogenase 4 |
| | | | | | | | | | | | | family, member A1 (ALDH4A1), |
| | | | | | | | | | | | | nuclear gene encoding |
| | | | | | | | | | | | | mitochondrial protein, transcript |
| | | | | | | | | | | | | variant P5CDhS, mRNA. |
| | 202148_s_at | PYCR1; | NM_006907 | 17q25.3 | Hs.79217 | 1.12087 | Up | 0.121682507 | 1.1226109 | Up | 0.08014123 | synonyms: P5C, P5CR, PYCR, |
| | | PYCR1; P5C; | | | | | | | | | | PP222; P5C reductase; Homo |
| | | P5CR; PP222 | | | | | | | | | | sapiens pyrroline-5-carboxylate |
| | | | | | | | | | | | | reductase 1 (PYCR1), nuclear |
| | | | | | | | | | | | | gene encoding mitochondrial |
| | | | | | | | | | | | | protein, transcript variant 1, |
| | | | | | | | | | | | | mRNA.; synonyms: P5C, P5CR, |
| | | | | | | | | | | | | PYCR, PP222; P5C reductase; |
| | | | | | | | | | | | | Homo sapiens pyrroline-5- |
| | | | | | | | | | | | | carboxylate reductase 1 |
| | | | | | | | | | | | | (PYCR1), nuclear gene |
| | | | | | | | | | | | | encoding mitochondrial protein, |
| | | | | | | | | | | | | transcript variant 2, mRNA. |
| metallo proteins | 208581_x_at | MT1x | NM_005952 | 16q13 | Hs.374950 | 1.547521 | Up | 0.010241902 | 1.3210147 | Up | 0.0043797 | synonyms: MT1, MT [symbol] Homo |
| [symbol] | | | | | | | | | | | | sapiens metallothionein 1X |
| | | | | | | | | | | | | (MT1X), mRNA. |
| | 204326_x_at | MT1L | NM_002450 | 16q13 | Hs.380778 | 1.623265 | Up | 0.017017261 | 1.232518 | Up | 0.0251237 | synonyms: MT1, MT1R; |
| | | | | | | | | | | | | metallothionein 1R; Homo |
| | | | | | | | | | | | | sapiens metallothionein 1L |
| | | | | | | | | | | | | (MT1L), mRNA. |
| | 204745_x_at | MT1G; MT1G: | NM_005950 | 16q13 | Hs.433391 | 1.26968 | Up | 0.0322672 | 1.364578 | Up | 0.0359903 | synonyms: MT1, MGC12386; |
| | | MGC12386 | | | | | | | | | | Homo sapiens metallothionein |
| | | | | | | | | | | | | 1G (MT1G), mRNA. |
| | 206461_x_at | MT1H | NM_005951 | 16q13 | Hs.2667 | 1.365586 | Up | 0.025688532 | 1.3155074 | Up | 0.0133935 | synonym: MT1; Homo sapiens |
| | | | | | | | | | | | | metallothionein 1H (MT1H), |
| | | | | | | | | | | | | mRNA. |
| | 211456_x_at | | AF333388 | | Hs.367850 | 1.354488 | Up | 0.018339171 | 1.3367926 | Up | 0.0097776 | MT-1H-like protein; mutant as |
| | | | | | | | | | | | | compared to wild-type sequence |
| | | | | | | | | | | | | MT-1H in GenBank Accession |
| | | | | | | | | | | | | Number X64834; Homo sapiens |
| | | | | | | | | | | | | metallothionein 1H-like protein |
| | | | | | | | | | | | | mRNA, complete cds. |
| | 212185_x_at | MT2A | NM_005953 | 16q13 | Hs.118786 | 1.364172 | Up | 0.033675359 | 1.3559748 | Up | 0.0171696 | synonym: MT2; This sequence |
| | | | | | | | | | | | | comes from FIG. 2; Homo |
| | | | | | | | | | | | | sapiens metallothionein 2A |
| | | | | | | | | | | | | (MT2A), mRNA. |
| | 212859_x_at | MT1E | BF217861 | 16q13 | Hs.433205 | 1.34909 | Up | 0.012855859 | 1.2187557 | Up | 0.023793 | metallothionein 1E (functional) |
| | 217165_x_at | MT1F; MT1F; | M10943 | 16q13 | | 1.186389 | Up | 0.226324931 | 1.2547997 | Up | 0.0079279 | synonyms: MT1, MGC32732; |
| | | MGC32732 | | | | | | | | | | Homo sapiens metallothionein |
| | | | | | | | | | | | | 1F (functional) (MT1F), mRNA. |
| | 213629_x_at | MT1F | BF246115 | 16q13 | Hs.381097 | 1.120311 | Up | 0.312657589 | 1.1835955 | Up | 0.057089 | metallothionein 1F (functional) |
| Ornithine related | 201599_at | OAT; OAT; | NM_000274 | 10q26 | Hs.75485 | 1.210476 | Down | 0.003978229 | 0.8746633 | Down | 0.01912681 | synonym: HOGA; Ornithine |
| | | HOGA | | | | | | | | | | aminotransferase; Homo |
| | | | | | | | | | | | | sapiens ornithine |
| | | | | | | | | | | | | aminotransferase (gyrate |
| | | | | | | | | | | | | atrophy) (OAT), nuclear gene |
| | | | | | | | | | | | | encoding mitochondrial protein, |
| | | | | | | | | | | | | mRNA. |
| | 212461_at | OAZIN | BF793951 | 8q22.3 | Hs.223014 | 1.239532 | Down | 0.016483367 | 1.1311716 | Down | 0.0150402 | ornithine decarboxylase |
| | | | | | | | | | | | | antizyme inhibitor |
| | 201364_s_at | OAZ2 | AF242521 | 15q22.1 | Hs.74563 | 1.115702 | Down | 0.079601378 | 1.1385592 | Down | 0.0359062 | protein translation dependent on |
| | | | | | | | | | | | | +1 ribosomal frameshift; |
| | | | | | | | | | | | | antizyme 2; Homo sapiens |
| | | | | | | | | | | | | ornithine decarboxylase |
| | | | | | | | | | | | | antizyme 2 (OAZ2), mRNA. |
| Arginine Related | 203946_s_at | ARG2 | U75667 | 14q24.1-q24.3 | Hs.17285 | 1.241001 | Down | 0.006993509 | 1.1794939 | Down | 0.0060619 | kidney arginase nonhepatic |
| [symbol] | | | | | | | | | | | | arginase L-arginine |
| | | | | | | | | | | | | amino hydrolase L-arginine |
| | | | | | | | | | | | | ureahydrolase A [symbol] Homo |
| | | | | | | | | | | | | sapiens arginase type |
| | | | | | | | | | | | | (ARG2) nuclear gene encoding |
| | | | | | | | | | | | | mitochondrial protein mRNA |
| | 202262_x_at | DDAH2; | NM_013974 | 6p21.3 | Hs.247362 | 1.218874 | Up | 0.045169089 | 1.1140322 | Up | 0.06574631 | synonyms: G6a, NG30, DDAHII; |
| | | DDAH2; G6a; | | | | | | | | | | dimethylarginine |
| | | NG30; DDAHII | | | | | | | | | | dimethylaminohydrolase II; |
| | | | | | | | | | | | | Homo sapiens dimethylarginine |
| | | | | | | | | | | | | dimethylaminohydrolase 2 |
| | | | | | | | | | | | | (DDAH2), mRNA. |
| ATP synthase | 201089_at | ATP6V1B2; | NM_001693 | 8p22-p21 | Hs.1697 | 1.203827 | Down | 0.006759781 | 0.8220404 | Down | 0.00155735 | synonyms: HO57, VATB, VPP3, |
| | | ATP6V1B2; | | | | | | | | | | Vma2, ATP6B2, ATP6B1B2; |
| | | HO57; VATB; | | | | | | | | | | vacuolar proton pump B isoform |
| | | VPP3; Vma2; | | | | | | | | | | 2; endomembrane proton pump |
| | | ATP6B2; | | | | | | | | | | 58 kDa subunit; vacuolar ATP |
| | | ATP6B1B2 | | | | | | | | | | synthase subunit B, brain |
| | | | | | | | | | | | | isoform; V-ATPase B2 subunit; |
| | | | | | | | | | | | | H(+)-transporting two-sector |
| | | | | | | | | | | | | ATPase, 56/58 kD subunit, |
| | | | | | | | | | | | | isoform 2; Homo sapiens |
| | | | | | | | | | | | | ATPase, H+ transporting, |
| | | | | | | | | | | | | lysosomal 56/58 kDa, V1 subunit |
| | | | | | | | | | | | | B, isoform 2 (ATP6V1B2), |
| | | | | | | | | | | | | mRNA. |
| (mitochondrial) | 201443_s_at | ATP6IP2; | AF248966 | Xq21 | Hs.183434 | 1.138442 | Down | 0.019987098 | 0.8876037 | Down | 0.00772083 | synonyms: M8-9, APT6M8-9, |
| | | ATP6IP2; M8-9; | | | | | | | | | | ATP6M8-9; ATPase, H+ |
| | | APT6M8-9; | | | | | | | | | | transporting, lysosomal |
| | | ATP6M8-9 | | | | | | | | | | (vacuolar proton pump) |
| | | | | | | | | | | | | membrane sector associated |
| | | | | | | | | | | | | protein M8-9; vacuolar ATP |
| | | | | | | | | | | | | synthase membrane sector |
| | | | | | | | | | | | | associated protein M8-9; V- |
| | | | | | | | | | | | | ATPase M8.9 subunit; ATPase |
| | | | | | | | | | | | | membrane sector associated |
| | | | | | | | | | | | | protein M8-9; renin receptor; |
| | | | | | | | | | | | | Homo sapiens ATPase, H+ |
| | | | | | | | | | | | | transporting, lysosomal |
| | | | | | | | | | | | | interacting protein 2 (ATP6IP2), |
| | | | | | | | | | | | | mRNA. |
| | 201444_s_at | ATP6IP2; | NM_005765 | Xq21 | Hs.183434 | 1.302733 | Down | 0.012483331 | 0.7879111 | Down | 0.01120869 | synonyms: M8-9, APT6M8-9, |
| | | ATP6IP2; M8-9; | | | | | | | | | | ATP6M8-9; ATPase, H+ |
| | | APT6M8-9; | | | | | | | | | | transporting, lysosomal |
| | | ATP6M8-9 | | | | | | | | | | (vacuolar proton pump) |
| | | | | | | | | | | | | membrane sector associated |
| | | | | | | | | | | | | protein M8-9; vacuolar ATP |
| | | | | | | | | | | | | synthase membrane sector |
| | | | | | | | | | | | | associated protein M8-9; V- |
| | | | | | | | | | | | | ATPase M8.9 subunit; ATPase |
| | | | | | | | | | | | | membrane sector associated |
| | | | | | | | | | | | | protein M8-9; renin receptor; |
| | | | | | | | | | | | | Homo sapiens ATPase, H+ |
| | | | | | | | | | | | | transporting, lysosomal |
| | | | | | | | | | | | | interacting protein 2 (ATP6IP2), |
| | | | | | | | | | | | | mRNA. |
| | 202874_s_at | ATP6V1C1; | NM_001695 | 8q22.3 | Hs.86905 | 1.244045 | Down | 0.012519633 | 0.8194812 | Down | 0.01590041 | synonyms: VATC, Vma5, |
| | | ATP6V1C1; | | | | | | | | | | ATP6C, ATP6D, FLJ20057; |
| | | VATC; Vma5; | | | | | | | | | | vacuolar proton-ATPase, |
| | | ATP6C; ATP6D; | | | | | | | | | | subunit C, VI domain; H+- |
| | | FLJ20057 | | | | | | | | | | transporting ATPase chain C, |
| | | | | | | | | | | | | vacuolar; vacuolar proton pump |
| | | | | | | | | | | | | C subunit; H(+)-transporting two- |
| | | | | | | | | | | | | sector ATPase, subunit C; |
| | | | | | | | | | | | | vacuolar ATP synthase subunit |
| | | | | | | | | | | | | C; V-ATPase C subunit; |
| | | | | | | | | | | | | vacuolar proton pump, 42-kD |
| | | | | | | | | | | | | subunit; vat c; H+-ATPase C |
| | | | | | | | | | | | | subunit; ATPase, H+ |
| | | | | | | | | | | | | transporting, lysosomal, 42 kD; |
| | | | | | | | | | | | | ATPase, H+ transporting, |
| | | | | | | | | | | | | lysosomal, subunit C; Homo |
| | | | | | | | | | | | | sapiens ATPase, H+ |
| | | | | | | | | | | | | transporting, lysosomal 42 kDa, |
| | | | | | | | | | | | | V1 subunit C, isoform 1 |
| | | | | | | | | | | | | (ATP6V1C1), mRNA. |
| ATP synthase | 202325_s_at | ATP5J; ATP5J; | NM_001685 | 21q21.1 | Hs.73851 | 1.196518 | Down | 0.00068097 | 0.8946735 | Down | 0.00601154 | synonyms: ATP5, ATPM, |
| | | ATPM; ATP5A | | | | | | | | | | ATP5A; ATP synthase, H+ |
| | | | | | | | | | | | | transporting (ATPase, |
| | | | | | | | | | | | | mitochondrial); ATP synthase |
| | | | | | | | | | | | | coupling factor 6; Homo sapiens |
| | | | | | | | | | | | | ATP synthase, H+ transporting, |
| | | | | | | | | | | | | mitochondrial F0 complex, |
| | | | | | | | | | | | | subunit F6 (ATP5J), nuclear |
| | | | | | | | | | | | | gene encoding mitochondrial |
| | | | | | | | | | | | | protein, mRNA. |
| (vaculolar) | 207507_s_at | ATP5G3 | NM_001689 | 2q31.1 | Hs.429 | 1.193118 | Down | 0.005733489 | 0.8544746 | Down | 0.00014404 | ATP synthase, mitochondrial, C |
| | | | | | | | | | | | | subunit-3; Homo sapiens ATP |
| | | | | | | | | | | | | synthase, H+ transporting, |
| | | | | | | | | | | | | mitochondrial F0 complex, |
| | | | | | | | | | | | | subunit c (subunit 9) isoform 3 |
| | | | | | | | | | | | | (ATP5G3), mRNA. |
| | 207508_at | ATP5G3 | NM_001689 | 2q31.1 | Hs.429 | 1.131765 | Down | 0.034321 | 0.8651774 | Down | 0.00238781 | ATP synthase, mitochondrial, C |
| | | | | | | | | | | | | subunit-3; Homo sapiens ATP |
| | | | | | | | | | | | | synthase, H+ transporting, |
| | | | | | | | | | | | | mitochondrial F0 complex, |
| | | | | | | | | | | | | subunit c (subunit 9) isoform 3 |
| | | | | | | | | | | | | (ATP5G3), mRNA. |
| | 208745_at | ATP5L | AA917672 | 11q23 | Hs.107476 | 1.187875 | Down | 0.011545567 | 0.8447284 | Down | 0.00101299 | ATP synthase, H+ transporting, |
| | | | | | | | | | | | | mitochondrial F0 complex, |
| | | | | | | | | | | | | subunit g |
| | 208870_x_at | ATP5C1; | BC000931 | 10q22-q23 | Hs.155433 | 1.124441 | Down | 0.019343046 | 0.9124323 | Down | 0.01392037 | synonyms: ATP5C, ATP5CL1; |
| | | ATP5C1; | | | | | | | | | | H(heart)-type ATP synthase |
| | | ATP5CL1 | | | | | | | | | | gamma-subunit; ATP synthase, |
| | | | | | | | | | | | | H+ transporting, mitochondrial |
| | | | | | | | | | | | | F1 complex, gamma polypeptide- |
| | | | | | | | | | | | | like 1; Homo sapiens ATP |
| | | | | | | | | | | | | synthase, H+ transporting, |
| | | | | | | | | | | | | mitochondrial F1 complex, |
| | | | | | | | | | | | | gamma polypeptide 1 |
| | | | | | | | | | | | | (ATP5C1), mRNA. |
| | 211755_s_at | ATP5F1 | BC005960 | 1p13.1 | Hs.61634 | 1.162583 | Down | 0.003380778 | 0.8787983 | Down | 0.00153431 | ATP synthase, H+ transporting, |
| | | | | | | | | | | | | mitochondrial F0 complex, |
| | | | | | | | | | | | | subunit b, isoform 1 |
| | 213738_s_at | ATP5A1 | AI587323 | 18q12-q21 | Hs.405985 | 1.144936 | Down | 0.009591883 | 0.8790464 | Down | 0.00385739 | ATP synthase, H+ transporting, |
| | | | | | | | | | | | | mitochondrial F1 complex, alpha |
| | | | | | | | | | | | | subunit, isoform 1, cardiac |
| | | | | | | | | | | | | muscle |
| Complex 1 | 201304_at | NDUFA5; | NM_005000 | 7q32 | Hs.83916 | 1.251252 | Down | 0.004319206 | 0.8638455 | Down | 0.01954249 | synonyms: B13, NUFM, |
| | | NDUFA5; B13; | | | | | | | | | | UQOR13, FLJ12147, CI-13 KD- |
| | | NUFM; | | | | | | | | | | B; NADH dehydrogenase |
| | | UQOR13; | | | | | | | | | | (ubiquinone) 1 alpha |
| | | FLJ12147; CI- | | | | | | | | | | subcomplex, 5 (13 kD, B13); |
| | | 13 KD-B | | | | | | | | | | Complex I-13 KD-B; ubiquinone |
| | | | | | | | | | | | | reductase; type I |
| | | | | | | | | | | | | dehydrogenase; Homo sapiens |
| | | | | | | | | | | | | NADH dehydrogenase |
| | | | | | | | | | | | | (ubiquinone) 1 alpha |
| | | | | | | | | | | | | subcomplex, 5, 13 kDa |
| | | | | | | | | | | | | (NDUFA5), nuclear gene |
| | | | | | | | | | | | | encoding mitochondrial protein, |
| | | | | | | | | | | | | mRNA. |
| | 202001_s_at | NDUFA6; | NM_002490 | 22q13.2-q13.31 | Hs.274416 | 1.190897 | Down | 0.002835277 | 0.8364088 | Down | 0.00058884 | synonym: B14; NADH |
| | | NDUFA5; B14 | | | | | | | | | | dehydrogenase (ubiquinone) 1 |
| | | | | | | | | | | | | alpha subcomplex, 6; NADH |
| | | | | | | | | | | | | dehydrogenase (ubiquinone) 1 |
| | | | | | | | | | | | | alpha subcomplex, 6 (14 kD, |
| | | | | | | | | | | | | B14); Homo sapiens NADH |
| | | | | | | | | | | | | dehydrogenase (ubiquinone) 1 |
| | | | | | | | | | | | | alpha subcomplex, 6, 14 kDa |
| | | | | | | | | | | | | (NDUFA6), mRNA. |
| | 202077_at | NDUFAB1; | NM_005003 | | Hs.5556 | 1.178986 | Down | 0.001418435 | 0.8300824 | Down | 0.00023024 | synonym: SDAP; NDUFAB1 |
| | | NDUFAB1; | | | | | | | | | | subunit; NADH dehydrogenase |
| | | SDAP | | | | | | | | | | (ubiquinone) 1, alpha/beta |
| | | | | | | | | | | | | subcomplex, 1 (8 kD, SDAP); |
| | | | | | | | | | | | | Homo sapiens NADH |
| | | | | | | | | | | | | dehydrogenase (ubiquinone) 1, |
| | | | | | | | | | | | | alpha/beta subcomplex, 1, 8 kDa |
| | | | | | | | | | | | | (NDUFAB1), mRNA. |
| | 203371_s_at | NDUFB3; | NM_002491 | 2q31.3 | Hs.109760 | 1.193323 | Down | 0.007489942 | 0.83959 | Down | 0.00103955 | synonym: B12; NADH |
| | | NDUFB3; B12 | | | | | | | | | | dehydrogenase (ubiquinone) 1 |
| | | | | | | | | | | | | beta subcomplex, 3 (12 kD, |
| | | | | | | | | | | | | B12); Homo sapiens NADH |
| | | | | | | | | | | | | dehydrogenase (ubiquinone) 1 |
| | | | | | | | | | | | | beta subcomplex, 3, 12 kDa |
| | | | | | | | | | | | | (NDUFB3), mRNA. |
| | 203613_s_at | NDUFB6; | NM_002493 | | Hs.109646 | 1.138507 | Down | 0.022389242 | 0.8259696 | Down | 0.00038684 | synonym: B17; NADH |
| | | NDUFB6; B17 | | | | | | | | | | dehydrogenase (ubiquinone) 1 |
| | | | | | | | | | | | | beta subcomplex, 6 (17 kD, |
| | | | | | | | | | | | | B17); Homo sapiens NADH |
| | | | | | | | | | | | | dehydrogenase (ubiquinone) 1 |
| | | | | | | | | | | | | beta subcomplex, 6, 17 kDa |
| | | | | | | | | | | | | (NDUFB6), mRNA. |
| | 203621_at | NDUFB5; | NM_002492 | 3q27.1 | Hs.19236 | 1.151684 | Down | 0.042430346 | 0.8951766 | Down | 0.01913304 | synonym: SGDH; NADH |
| | | NDUFB5; | | | | | | | | | | dehydrogenase (ubiquinone) 1 |
| | | SGDH | | | | | | | | | | beta subcomplex, 5 (16 kD, |
| | | | | | | | | | | | | SGDH); Homo sapiens NADH |
| | | | | | | | | | | | | dehydrogenase (ubiquinone) 1 |
| | | | | | | | | | | | | beta subcomplex, 5, 16 kDa |
| | | | | | | | | | | | | (NDUFB5), mRNA. |
| | 206790_s_at | NDUFB1; | NM_004545 | 14q32.12 | Hs.183435 | 1.190237 | Down | 0.004969015 | 0.8718049 | Down | 0.00309552 | synonyms: MNLL, CI-SGDH; |
| | | NDUFB1; | | | | | | | | | | NADH dehydrogenase |
| | | MNLL; CI- | | | | | | | | | | (ubiquinone) 1 beta subcomplex, |
| | | SGDH | | | | | | | | | | 1 (7 kD, MNLL); Homo sapiens |
| | | | | | | | | | | | | NADH dehydrogenase |
| | | | | | | | | | | | | (ubiquinone) 1 beta subcomplex, |
| | | | | | | | | | | | | 1, 7 kDa (NDUFB1), mRNA. |
| | 209303_at | NDUFS4; | BC005270 | 5q11.1 | Hs.10758 | 1.15407 | Down | 0.02314134 | 0.833423 | Down | 0.00049245 | synonym: AQDQ; NADH |
| | | NDUFS4; | | | | | | | | | | dehydrogenase (ubiquinone) Fe— |
| | | AQDQ | | | | | | | | | | S protein 4 (18 kD) (NADH- |
| | | | | | | | | | | | | coenzyme Q reductase); NADH |
| | | | | | | | | | | | | dehydrogenase (ubiquinone) Fe— |
| | | | | | | | | | | | | S protein 4, 18 kD (NADH- |
| | | | | | | | | | | | | coenzyme Q; mitochondrial |
| | | | | | | | | | | | | respiratory chain complex I (18- |
| | | | | | | | | | | | | KD subunit); Homo sapiens |
| | | | | | | | | | | | | NADH dehydrogenase |
| | | | | | | | | | | | | (ubiquinone) Fe—S protein 4, |
| | | | | | | | | | | | | 18 kDa (NADH-coenzyme Q |
| | | | | | | | | | | | | reductase) (NDUFS4), mRNA. |
| | 217773_s_at | NDUFA4; | NM_002489 | | Hs.50098 | 1.185965 | Down | 0.005342988 | 0.869414 | Down | 0.00144196 | synonym: MLRQ; NADH |
| | | NDUFA4; | | | | | | | | | | dehydrogenase (ubiquinone) 1 |
| | | MLRQ | | | | | | | | | | alpha subcomplex, 4 (9 kD, |
| | | | | | | | | | | | | MLRQ); Homo sapiens NADH |
| | | | | | | | | | | | | dehydrogenase (ubiquinone) 1 |
| | | | | | | | | | | | | alpha subcomplex, 4, 9 kDa |
| | | | | | | | | | | | | (NDUFA4), mRNA. |
| | 218101_s_at | NDUFC2; | NM_004549 | | Hs.193313 | 1.129474 | Down | 0.031751159 | 0.8886449 | Down | 0.00161606 | synonym: B14.5b; NADH |
| | | NDUFC2; | | | | | | | | | | dehydrogenase (ubiquinone) 1, |
| | | B14.5b | | | | | | | | | | subcomplex unknown, 2 |
| | | | | | | | | | | | | (14.5 kD, B14.5b); Homo sapiens |
| | | | | | | | | | | | | NADH dehydrogenase |
| | | | | | | | | | | | | (ubiquinone) 1, subcomplex |
| | | | | | | | | | | | | unknown, 2, 14.5 kDa |
| | | | | | | | | | | | | (NDUFC2), mRNA. |
| | 218226_s_at | NDUFB4; | NM_004547 | 3q13.33 | Hs.227750 | 1.118038 | Down | 0.018501169 | 0.9263017 | Down | 0.03138745 | synonym: B15; NDUFB4 |
| | | NDUFB4; B15 | | | | | | | | | | subunit; NADH dehydrogenase |
| | | | | | | | | | | | | (ubiquinone) 1 beta subcomplex, |
| | | | | | | | | | | | | 4 (15 kD, B15); Homo sapiens |
| | | | | | | | | | | | | NADH dehydrogenase |
| | | | | | | | | | | | | (ubiquinone) 1 beta subcomplex, |
| | | | | | | | | | | | | 4, 15 kDa (NDUFB4), mRNA. |
| Complex 3 | 4902233_s_at | UQGRH | NM_006004 | | Hs.73818 | 1.148508 | Down | 0.025106608 | 0.8560056 | Down | 3.687E−05 | Homo sapiens ubiquinol- |
| | | | | | | | | | | | | cytochrome c reductase hinge |
| | | | | | | | | | | | | protein (UQGRH) mRNA |
| | 208909_at | UQCRPS | B0000649 | 9q12-q3.1 | Hs.3712 | 1.168573 | Down | 0.012592794 | 0.8267155 | Down | 0.0002244 | synonym RIS1 Homo sapiens |
| | | UQCRPS | | | | | | | | | | ubiquinol-cytochrome c |
| | | RIS1 | | | | | | | | | | reductase Rlesed non-sulfu [symbol] |
| | | | | | | | | | | | | polypeptide 1 (UQCR [symbol] S1) |
| | | | | | | | | | | | | nuclear gene encoding |
| | | | | | | | | | | | | mitochondrial protein, mRNA. |
| | 200883_at | UQCRC2 | NM_003366 | 16p12 | Hs.173554 | 1.213298 | Down | 0.00595247 | 0.6553051 | Down | 0.01987364 | Homo sapiens ubiquinol- |
| | | | | | | | | | | | | cytochrome c reductase core |
| | | | | | | | | | | | | protein II (UQCRC2), mRNA. |
| | 205849_s_at | UQCRB; | NM_006294 | 8q22 | Hs.131255 | 1.185495 | Down | 0.012369038 | 0.8808683 | Down | 0.0017732 | synonyms: QPC, QP-C, UQBC, |
| | | UQCRB; QPC; | | | | | | | | | | UQBP, UQPC; Homo sapiens |
| | | QP-C; UQBC; | | | | | | | | | | ubiquinol-cytochrome c |
| | | UQBP; UQPC | | | | | | | | | | reductase binding protein |
| | | | | | | | | | | | | (UQCRB), mRNA. |
| | 212600_s_at | UQCRC2 | AV727381 | 16p12 | Hs.173554 | 1.116486 | Down | 0.039363536 | 0.8832586 | Down | 0.00798426 | ubiquinol-cytochrome c |
| | | | | | | | | | | | | reductase core protein II |
| Complex 4 | 201597_at | COX7A2 | NM_001865 | 6q12 | Hs.70312 | 1.149356 | Down | 0.007964768 | 0.8467176 | Down | 184E−05 | synonyms COX7AL, COX7AL1 |
| | | COX7A2 | | | | | | | | | | COXVIIa-L hepatic cytochrome |
| | | COX7AL | | | | | | | | | | c oxidase chain VIIa Homo |
| | | COX7AL1 | | | | | | | | | | sapiens cytochrome c oxidase |
| | | COXVIIa-L | | | | | | | | | | subunit VIIa polypeptide 2 (liver) |
| | | | | | | | | | | | | (COX7A2) nuclear gene |
| | | | | | | | | | | | | encoding mitochondrial protein, |
| | | | | | | | | | | | | mRNA. |
| | 202110_at | COX7B | NM_001866 | Xq13.2 | Hs.432170 | 1.130844 | Down | 0.045735159 | 0.8529508 | Down | 0.00061257 | cytochrome-c oxidase chain |
| | | | | | | | | | | | | VIIb; Homo sapiens cytochrome |
| | | | | | | | | | | | | c oxidase subunit VIIb (COX7B), |
| | | | | | | | | | | | | nuclear gene encoding |
| | | | | | | | | | | | | mitochondrial protein, mRNA. |
| | 203663_s_at | COX5A | NM_004255 | 15q25 | Hs.32383 | 1.197943 | Down | 0.004379832 | 0.8133834 | Down | 7.2462E−05 | synonyms-VA COX COX-VA |
| | | COX6A VA | | | | | | | | | | cytochrome c oxidase |
| | | COX6A | | | | | | | | | | polypeptide mitochondrial |
| | | | | | | | | | | | | precursor Homo sapiens |
| | | | | | | | | | | | | cytochrome c oxidase subunit |
| | | | | | | | | | | | | Va (COX5A), nuclear gene |
| | | | | | | | | | | | | encoding mitochondrial protein |
| | | | | | | | | | | | | mRNA. |
| | 203880_at | COX17 | NM_005694 | | Hs.16297 | 1.199099 | Down | 0.014912612 | 0.8134965 | Down | 0.00016529 | human homolog of yeast |
| | | | | | | | | | | | | mitochondrial copper |
| | | | | | | | | | | | | recruitment gene; COX17 |
| | | | | | | | | | | | | (yeast) homolog, cytochrome c |
| | | | | | | | | | | | | oxidase assembly protein; Homo |
| | | | | | | | | | | | | sapiens COX17 homolog, |
| | | | | | | | | | | | | cytochrome c oxidase assembly |
| | | | | | | | | | | | | protein (yeast) (COX17), nuclear |
| | | | | | | | | | | | | gene encoding mitochondrial |
| | | | | | | | | | | | | protein, mRNA. |
| | 214277_at | COX11 | AI376724 | 17q22 | Hs.241515 | 1.287688 | Down | 0.009150653 | 0.8605178 | Down | 0.02984133 | COX11 homolog, cytochrome c |
| | | | | | | | | | | | | oxidase assembly protein |
| | | | | | | | | | | | | (yeast) |
| | 217491_x_at | COX7CP1 | AF042165 | 13q14-q21 | | 1.148912 | Down | 0.031232864 | 0.9439594 | Down | 0.04448096 | cytochrome c oxidase subunit |
| | | | | | | | | | | | | VIIc; E.C. number = 1.9.3.1; |
| | | | | | | | | | | | | Homo sapiens cytochrome c |
| | | | | | | | | | | | | oxidase subunit VIIc |
| | | | | | | | | | | | | (COX7CP1) pseudogene, |
| | | | | | | | | | | | | complete sequence. |
| | 217329_x_at | COX7BP1; | AF042164 | 22q13 | | 1.226784 | Down | 0.002837439 | 1.1261074 | Down | 0.06248038 | cytochrome c oxidase subunit |
| | | bK714B7.1 | | | | | | | | | | VIIb; E.C. number = 1.9.3.1; |
| | | | | | | | | | | | | Homo sapiens cytochrome c |
| | | | | | | | | | | | | oxidase subunit VIIb |
| | | | | | | | | | | | | (COX7BP1) pseudogene, |
| | | | | | | | | | | | | complete sequence. |
| Holocytochrome | 203745_at | HCCS | AI801013 | Xp22.3 | Hs.211571 | 1.168718 | Down | 0.002771726 | 0.8395424 | Down | 0.00635825 | holocytochrome c synthase |
| c Synthetase | | | | | | | | | | | | (cytochrome c heme-lyase) |
| | 203746_s_at | HCCS; HCCS; | NM_005333 | Xp22.3 | Hs.211571 | 1.192373 | Down | 0.005742037 | 0.9000634 | Down | 0.01929721 | synonym: CCHL; putative; |
| | | CCHL | | | | | | | | | | Homo sapiens holocytochrome c |
| | | | | | | | | | | | | synthase (cytochrome c heme- |
| | | | | | | | | | | | | lyase) (HCCS), mRNA. |
| Adenine | 202825_at | SLC25A4 | NM_001161 | 4q35 | Hs.2043 | 1.243055 | Down | 0.003755192 | 0.8545047 | Down | 0.00413595 | synonyms: T1, ANT ANT 1 |
| translocators | | SLC25A4 T | | | | | | | | | | PEO2 PEO3 adenine |
| | | ANT ANT 1 | | | | | | | | | | nucleotide translocator 1 |
| | | PEO2 PEO3 | | | | | | | | | | (skeletal muscle), Homo sapiens |
| | | | | | | | | | | | | solute carrier family 25 |
| | | | | | | | | | | | | (mitochondrial carrier adenine |
| | | | | | | | | | | | | nucleotide translocator) |
| | | | | | | | | | | | | member 4 (SLC25A4) nuclear |
| | | | | | | | | | | | | gene encoding mitochondrial |
| | | | | | | | | | | | | protein mRNA. |
| Glycine/Serine | 204294_at | AMT; AMT; | NM_000481 | 3p21.2-p21.1 | Hs.102 | 1.20989 | Up | 0.003991538 | 1.0981119 | Up | 0.01028424 | synonyms: GCE, NKH, GCST; |
| metabolism | | GCE; NKH; | | | | | | | | | | Homo sapiens |
| | | GCST | | | | | | | | | | aminomethyltransferase (glycine |
| | | | | | | | | | | | | cleavage system protein T) |
| | | | | | | | | | | | | (AMT), mRNA. |
| Voltage | 211662_s_at | VDAC2 | L08666 | 10q22 | Hs.78902 | 1.12617 | Down | 0.014133989 | 0.8896027 | Down | 0.00348693 | Homo sapiens voltage |
| dependent | | | | | | | | | | | | dependent anion channel 2 |
| anion channels | | | | | | | | | | | | (VDAC2) mRNA. |
| (In mitochondrial- | 217140_s_at | VDAC1P | AJ002428 | Xq21-q22 | | 1.215466 | Down | 0.018102094 | 0.7707105 | Down | 0.0008464 | Homo sapiens VDAC1 |
| outer-membrane) | | | | | | | | | | | | pseudogene. |
| | 208846_s_at | VDAC3; | U90943 | 8p11.2 | Hs.7381 | 1.150067 | Down | 0.060742368 | 1.1200638 | Down | 0.0287343 | synonym: HD-VDAC3; similar to |
| | | VDAC3; HD- | | | | | | | | | | mouse VDAC 3; Homo sapiens |
| | | VDAC3 | | | | | | | | | | voltage-dependent anion |
| | | | | | | | | | | | | channel 3 (VDAC3), mRNA. |
| Lactate | 213564_x_at | LDHB | BE042354 | 12p12.2-p12.1 | Hs.234489 | 1.104586 | Down | 0.030076871 | 0.9133205 | Down | 0.00138614 | lactate dehydrogenase B |
| metabolism | 200650_s_at | LDHA; LDHA; | NM_005566 | 11p15.4 | Hs.2795 | 1.134901 | Down | 0.03774599 | 1.1884595 | Down | 0.000774 | synonym: LDH1; Homo sapiens |
| | | LDH1 | | | | | | | | | | lactate dehydrogenase A |
| | | | | | | | | | | | | (LDHA), mRNA. |
| | 201030_x_at | LDHB | NM_002300 | 12p12.2-p12.1 | Hs.234489 | 1.08367 | Down | 0.05927145 | 1.1007107 | Down | 0.0010265 | Homo sapiens lactate |
| | | | | | | | | | | | | dehydrogenase B (LDHB), |
| | | | | | | | | | | | | mRNA. |
| Isocitrate | 210014_x_at | IDH3B, IDH3B | AF023266 | 20p10 | Hs.155410 | 1.122301 | Down | 0.019203205 | 0.8919882 | Down | 0.01916003 | synonyms-H IDHB MGC903 |
| dehydrogenase | | H IDHB | | | | | | | | | | FLJ11043, isocitric |
| | | MGC903 | | | | | | | | | | dehydrogenase NAD+ specific |
| | | FLJ11043 | | | | | | | | | | isocitrate dehydrogenase beta |
| | | | | | | | | | | | | precursor, NAD+ specific |
| | | | | | | | | | | | | isocitrate dehydrogenase [symbol] |
| | | | | | | | | | | | | subunit, NAD+ specific IODH |
| | | | | | | | | | | | | isocitrate dehydrogenase, |
| | | | | | | | | | | | | NAD(+) specific mitochondrial |
| | | | | | | | | | | | | beta subunit Homo sapiens |
| | | | | | | | | | | | | isocitrate dehydrogenase [symbol] |
| | | | | | | | | | | | | (NAD+) beta (IDH3B) nuclear |
| | | | | | | | | | | | | gene encoding mitochondrial |
| | | | | | | | | | | | | protein transcript variant 1 |
| | | | | | | | | | | | | mRNA. |
| [symbol] | 240418_s_at | IDH3B-IDH3B | AF023265 | 20p13 | Hs.155410 | 1.157609 | Down | 0.009473713 | 0.8451559 | Down | 0.00368349 | synonyms H-IDHB MGG903 |
| | | HIDHB | | | | | | | | | | FLJ11043, isocitric |
| | | MGC903 | | | | | | | | | | dehydrogenase NAD+-specific |
| | | FLJ11043 | | | | | | | | | | isocitrate dehydrogenase beta |
| | | | | | | | | | | | | precursor NAD+-specific |
| | | | | | | | | | | | | isocitrate dehydrogenase or |
| | | | | | | | | | | | | subunit NAD+-specific ICDH [symbol] |
| | | | | | | | | | | | | isocitrate dehydrogenase |
| | | | | | | | | | | | | NAD(+)-specific mitochondrial |
| | | | | | | | | | | | | beta subunit Homo sapiens |
| | | | | | | | | | | | | isocitrate dehydrogenase 3 |
| | | | | | | | | | | | | (NAD+) beta (IDH3B) nuclear |
| | | | | | | | | | | | | gene encoding mitochondrial |
| | | | | | | | | | | | | protein transeript variant 1 |
| | | | | | | | | | | | | mRNA |
| | 202069_s_at | IDH3A | AI826060 | 15q251-q25.2 | Hs.250616 | 1.42986 | Down | 0.000598139 | 0.7369903 | Down | 0.00167327 | isocitrate dehydrogenase 3 |
| | | | | | | | | | | | | (NAD+) alpha |
| | 202070_s_at | IDH3A | NM_005530 | 15q25.1-q25.2 | Hs.250616 | 1.107756 | Down | 0.098643023 | 1.1153047 | Down | 0.0234165 | isocitrate dehydrogenase [NAD] |
| | | | | | | | | | | | | subunit alpha, mitochondrial; |
| | | | | | | | | | | | | NAD+-specific ICDH; NAD(H)- |
| | | | | | | | | | | | | specific isocitrate |
| | | | | | | | | | | | | dehydrogenase alpha subunit |
| | | | | | | | | | | | | precursor; isocitrate |
| | | | | | | | | | | | | dehydrogenase (NAD+) alpha |
| | | | | | | | | | | | | chain precursor; H-IDH alpha; |
| | | | | | | | | | | | | isocitric dehydrogenase; Homo |
| | | | | | | | | | | | | sapiens isocitrate |
| | | | | | | | | | | | | dehydrogenase 3 (NAD+) alpha |
| | | | | | | | | | | | | (IDH3A), nuclear gene encoding |
| | | | | | | | | | | | | mitochondrial protein, mRNA. |
| HMG related | 215568_x_at | HMGCL | AL031296 | 1p36.1-p35 | Hs.831 [symbol] | 1.283746 | Up | 3.92631E−05 | | | | synonym HL-3 hydroxy-3 |
| [symbol] | | HMGCL HL | | | | | | | | | | methylglutaryl-Coenzyme A |
| | | | | | | | | | | | | lyase 3-hydroxy-3h |
| | | | | | | | | | | | | methylglutaryl-Coenzyme A |
| | | | | | | | | | | | | lyase |
| | | | | | | | | | | | | (hydroxymethylglutar [symbol] |
| | | | | | | | | | | | | Homo sapiens 3 hydroxymethyl |
| | | | | | | | | | | | | 3-methylglutaryl-Coenzyme A |
| | | | | | | | | | | | | lyase |
| | | | | | | | | | | | | (hydroxymethylgluta [symbol] ) |
| | | | | | | | | | | | | (HMGCL) mRNA. |
| | 202540_s_at | HMGCR | NM_000859 | 5q13.3-q14 | Hs.11899 | 1.237843 | Down | 0.004508094 | 0.8558926 | Down | 0.02049312 | Homo sapiens 3-hydroxy-3- |
| | | | | | | | | | | | | methylglutaryl-Coenzyme A |
| | | | | | | | | | | | | reductase (HMGCR), mRNA. |
| Glutamate | 219933_s_at | GLRX2; GLRX2 | NM_016066 | 1q31.2-q31.3 | Hs.5054 | 1.214851 | Down | 0.000850749 | 0.8082611 | Down | 0.0001692 | synonym GRX2, |
| metabolism | | GRX2 | | | | | | | | | | thio [symbol] transterase; contains |
| [symbol] | | | | | | | | | | | | nuclear membrane localisation |
| | | | | | | | | | | | | CGI-133 protein Homo sapiens |
| | | | | | | | | | | | | glutaredoxin 2 (GLRX2) mRNA |
| Oxiderelated | 202017_at | EPHX1; EPHX1; | NM_000120 | 1q42.1 | Hs.89649 | 1.405022 | Up | 0.065587794 | 1.4155553 | Up | 0.0222559 | synonyms: MEH, EPHX, EPOX; |
| | | MEH; EPOX | | | | | | | | | | Epoxide hydroxylase 1, |
| | | | | | | | | | | | | microsomal (xenobiotic); Homo |
| | | | | | | | | | | | | sapiens epoxide hydrolase 1, |
| | | | | | | | | | | | | microsomal (xenobiotic) |
| | | | | | | | | | | | | (EPHX1), mRNA. |

1. A method for identifying a potential therapeutic agent for the prevention, treatment, or amelioration of schizophrenia, which comprises:
contacting a cell with a candidate therapeutic agent, or administering a candidate therapeutic agent to an organism;
determining whether expression of any of the following genes is altered in the cell or organism in response to the candidate therapeutic agent: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1; and
identifying the candidate as a potential therapeutic agent if expression of one or more of the genes is altered.

2. A method according to claim 1, wherein it is determined whether expression of any of the genes is altered by comparing the expression level of the gene or genes in the presence and absence of the candidate therapeutic agent.

3. A method according to claim 1, wherein the candidate is identified as a potential therapeutic agent if expression of one or more of the following genes is increased: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; or expression of one or more of the following genes is decreased: FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

4. A method according to claim 1, wherein the candidate is identified as a potential therapeutic agent if expression of the majority of the following genes is altered in response to the therapeutic agent: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; AMT; CLN3; ACOX1; G6PD; GCDH; COL5A1; NY-REN-24; HMGCL; TXNL2; SOD3; BCAT2; MT1X.

5. A method according to claim 4, wherein the candidate is identified as a potential therapeutic agent if expression of the majority of the genes is altered in the following ways: an increase in expression of: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; a decrease in expression of: FBS1; WFS1; AMT; CLN3; ACOX1; G6PD; GCDH; COL5A1; NY-REN-24; HMGCL; TXNL2; SOD3; BCAT2; MT1X.

6. A method according to claim 1, wherein it is determined whether expression of the gene or genes is altered in the prefrontal cortex of the organism.

7. A method according to claim 1, wherein the organism is a mouse or a rat.

8. A method according to claim 1, wherein the organism is an animal model for a neuropsychiatric disorder.

9. A method according to claim 1, wherein the cell is a neuronal cell.

10. A method according to claim 1, wherein a microarray is used to determine whether expression of the gene or genes is altered.

11. A method according to claim 1, which further comprises determining the side effects caused by administration of the candidate if it is identified as a potential therapeutic agent.

12. A screening assay to identify a potential schizophrenia therapeutic agent for the prevention, treatment, or amelioration of schizophrenia which comprises screening for a modulator of expression of any of the genes specified in claim 1 by: providing a system capable of expressing any of the genes specified in claim 1; maintaining the system under conditions for expression of the gene in the presence and absence of a candidate modulator of expression of the gene; and determining the expression level of the gene in the presence and absence of the candidate modulator.

13. A screening assay according to claim 12, which comprises screening for an upregulator of expression of any of the following: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

14. A screening assay according to claim 12, which comprises screening for a downregulator of expression of any of the following: FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

15. A screening assay to identify a potential schizophrenia therapeutic agent for the prevention, treatment, or amelioration of schizophrenia which comprises screening for a regulator of the activity of any of the proteins encoded by the genes specified in claim 1 by: contacting the protein with a candidate regulator and determining the activity of the protein in the presence and absence of the candidate regulator.

16. A screening assay according to claim 15, which comprises screening for an enhancer or activator of the activity of any of the following proteins: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP61P2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

17. A screening assay according to claim 15, which comprises screening for an inhibitor of the activity of any of the following proteins: FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

18. A screening assay to identify a potential schizophrenia therapeutic agent for the prevention, treatment, or amelioration of schizophrenia which comprises screening for a regulator of the interaction of any of the proteins encoded by the genes specified in claim 1 with a binding partner required for the biological effect of the protein by: contacting the protein with the binding partner in the presence of a candidate regulator, and determining binding of the protein to its binding partner in the presence and absence of the candidate regulator.

19. A screening assay according to claim 18, which comprises screening for an enhancer of the interaction of any of the following proteins with a binding partner required for the biological effect of the protein: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

20. A screening assay according to claim 18, which comprises screening for an inhibitor of the interaction of any of the following proteins with a binding partner required for the biological effect of the protein: FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

21. A screening assay to identify a potential schizophrenia therapeutic agent for the prevention, treatment, or amelioration of schizophrenia which comprises screening for a binding partner of any of the proteins encoded by the genes specified in claim 1 by: contacting the protein with a sample comprising a candidate binding partner, and determining whether the candidate binding partner binds to the protein.

22. A screening assay to identify a potential therapeutic agent for the prevention, treatment, or amelioration of schizophrenia comprising using any of the following proteins or nucleic acids:
(i) proteins encoded by the following genes: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1 E; MT1F; DDAH2; AMT; HMGCL; EPHX1; or ii) nucleic acid encoding any of the proteins of (i) above.

23. A screening assay using a regulator of expression of any of (i) of claim 22 to identify a potential therapeutic agent for the prevention, treatment, or amelioration of schizophrenia.

24. A screening assay using of a binding partner of any of (i) or (ii) of claim 22 to identify a potential therapeutic agent for the prevention, treatment, or amelioration of schizophrenia.

25. A screening assay using of an expression vector comprising nucleic acid encoding any of (i) of claim 22 to identify a potential therapeutic agent for the prevention, treatment, or amelioration of schizophrenia.

26. A screening assay using of a cell or cell line expressing nucleic acid encoding any of (i) of claim 22 in a screening assay to identify a potential therapeutic agent for the prevention, treatment, or amelioration of schizophrenia.

27. A screening assay according to claim 26, wherein the cell is a neural cell.

28. A screening assay according to claim 26, wherein the cell is an oligodendrocyte.

29. A recombinant mouse in which expression of a gene encoding any of (i) of claim 22 is altered compared with expression of the corresponding gene in normal mice.

30. A recombinant mouse according to claim 29 in which expression of two or more of the genes is altered.

31. A recombinant mouse according to claim 29 or 30, which is a knockout mouse for the gene or genes.

32. A recombinant mouse according to claim 29, wherein the mouse is used as an animal model for schizophrenia.

33. A screening assay to identify a potential therapeutic agent for the prevention, treatment, or amelioration of schizophrenia comprising a mouse according to claim 29 or cells obtained or derived from the mouse.

34. A method of determining the level of expression of the gene comprising using a binding partner of nucleic acid encoding any of the genes specified in claim 1, or of a protein encoded by any of the genes specified in claim 1.

35. A method according to claim 34, wherein the method is used for diagnosing whether a subject has, or is at risk of developing schizophrenia.

36. A method of diagnosing whether a subject has, or is at risk of developing schizophrenia, which comprises determining the level of any of the proteins of (i) of claim 22, or the expression level of a gene encoding any of the proteins of (i) of claim 22, in a biological sample obtained from the subject, or in a sample derived from a biological sample obtained from the subject.

37. A method according to claim 36, wherein the expression level of the majority of the following genes, or the levels of the majority of the proteins encoded by the following genes is determined: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1: FBS1; WFS1; AMT; CLN3; ACOX1; G6PD; GCDH; COL5A1; NY-REN-24; HMGCL; TXNL2; SOD3; BCAT2; MT1X.

38. A method according to claim 36, wherein the biological sample comprises a peripheral tissue or cell type in which the level of the protein, or the expression level of the gene, correlates with the level of the corresponding protein, or the expression level of the corresponding protein, in the prefrontal cortex.

39. A method according to claim 38, wherein the peripheral tissue or cell type comprises a blood cell.

40. A method according to claim 39, wherein the blood cell is a macrophage, a monocyte, a lymphocyte, an erythrocyte, a platelet, a leukocyte (either a neutrophil, an eosinophil, or a basophil; a lymphocyte, or a monocyte).

41. A method of prevention, treatment, or amelioration of schizophrenia which comprises increasing the level or activity of any of the following proteins in the brain (in particular the prefrontal cortex) of a subject in need of such prevention, treatment, or amelioration: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP6IP2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2.

42. A method of prevention, treatment, or amelioration of schizophrenia which comprises reducing the level or activity of any of the following proteins in the brain (in particular the prefrontal cortex) of a subject in need of such prevention, treatment, or amelioration: FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

43. A method of prevention, treatment, or amelioration of schizophrenia which comprises increasing the level or activity of the majority of the following proteins in the brain (in particular the prefrontal cortex) of a subject in need of such prevention, treatment, or amelioration: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; and reducing the level or activity of the majority of the following proteins in the brain (in particular the prefrontal cortex) of the subject: FBS1; WFS1; AMT; CLN3; ACOX1; G6PD; GCDH; COL5A1; NY-REN-24; HMGCL; TXNL2; SOD3; BCAT2; MT1X.

44. A microarray for use in a method for identifying a potential therapeutic agent according to claim 1 for the diagnosis, prevention, treatment, or amelioration of schizophrenia.

45. A microarray according to claim 44, which is a gene chip comprising a plurality of different probes capable of hybridising to nucleic acid expression products, or nucleic acid derived from nucleic acid expression products, of the majority of the following genes: PARG; VDAC2; OLR1; ARPC3; UQCRFS1; DNCLI1; PPM1A; ATPIF1; GLRX2; TIMM17A; IDH3B; ARG2; DNAJA1; SST; NEUROD6; ICAP-1A; FLJ23251; KCNK1; SLC25A4; FLJ13611; HIRIP5; COX7A2; COX5A; TAC1; UQCRH; MAGEH1; C13orf12; EBNA1BP2; DIRAS2; MPPE1; FBS1; WFS1; AMT; CLN3; ACOX1; G6PD; GCDH; COL5A1; NY-REN-24; HMGCL; TXNL2; SOD3; BCAT2; MT1X.

46. A microarray according to claim 44 wherein the microarray is used for identifying a potential therapeutic agent for the prevention, treatment, or amelioration of schizophrenia, or in a method of diagnosis of schizophrenia.

47. A kit for use in a method for identifying a potential therapeutic agent according to claim 1 for the diagnosis, prevention, treatment, or amelioration of schizophrenia wherein the kit comprises components for detecting expression products, or nucleic acids derived from nucleic acid expression products, of a plurality of the following genes: PARG; OLR1; ARPC3; DNCLI1; PPM1A; ATPIF1; TIMM17A; DNAJA1; SST; NEUROD6; ICAP-1A; FLJ23251; KCNK1; FLJ13611; HIRIP5; TAC1; MAGEH1; C13orf12; EBNA1BP2; DIRAS2; MPPE1; OAT; OAZIN; OAZ2; ARG2; ATP6V1B2; ATP61P2; ATP6V1C1; ATP5J; ATP5G3; ATP5L; ATP5C1; ATP5F1; ATP5A1; NDUFA5; NDUFA6; NDUFAB1; NDUFB3; NDUFB6; NDUFB5; NDUFB1; NDUFS4; NDUFA4; NDUFC2; NDUFB4; UQCRH; UQCRFS1; UQCRC2; UQCRB; UQCRC2; COX7A2; COX7B; COX5A; COX17; COX11; COX7CP1; COX7BP1; HCCS; SLC25A4; VDAC2; VDAC1P; VDAC3; LDHB; LDHA; IDH3B; IDH3A; HMGCR; GLRX2; FBS1; WFS1; AMT; CLN3; ACOX1; G6PD; GCDH; COL5A1; NY-REN-24; TXNL2; SOD3; BCAT2; ALDH4A1; PYCR1; MT1X; MT1L; MT1G; MT1H; MT2A; MT1E; MT1F; DDAH2; AMT; HMGCL; EPHX1.

48. A kit according to claim 47, which further comprises one or more of the following:
i) instructions for using the detecting means for diagnosis, prognosis, or therapeutic monitoring;
ii) a labelled moiety for detecting the detecting means;
iii) a solid phase to which the detecting means is immobilised;
iv) a predetermined amount of an isolated expression product of one or more of the genes for use as a standard, or control;
v) a label or insert indicating regulatory approval for diagnostic, prognostic or therapeutic use as appropriate.

49. A method for identifying a schizophrenia patient, or a patient suspected of having schizophrenia, who is likely to respond to a therapeutic treatment that alters the level or activity of any of the proteins specified in (i) of claim 22, the method comprising: determining the level of expression of any of the genes encoding the proteins specified in (i) of claim 22 in a patient, or in a biological sample obtained from the patient; and identifying the patient as being likely to respond to the therapeutic treatment if the level of expression of the gene or genes is altered compared to a normal subject.

50. A method for selecting a participant in a clinical trial to determine the effectiveness of a potential therapeutic agent for the prevention, treatment, or amelioration of schizophrenia, the method comprising: determining the level of expression of any of the genes encoding the proteins specified in (i) of claim 22 in a candidate participant, or in a biological sample obtained from the candidate participant; and selecting the candidate for the clinical trial if the level of expression of the gene or genes is altered compared to a normal subject.
